(19) United States
(12) Patent Application Publication
(10) Pub. No.: US 2024/0212370 A1
(43) Pub. Date: Jun. 27, 2024

(54) DATA ACQUISITION DEVICE, DATA ACQUISITION METHOD, AND BIOLOGICAL SAMPLE OBSERVATION SYSTEM

(71) Applicant: Sony Group Corporation, Tokyo (JP)

(72) Inventors: Tomohiko Nakamura, Tokyo (JP); Kazuhiro Nakagawa, Saitama (JP)

(73) Assignee: Sony Group Corporation, Tokyo (JP)

(21) Appl. No.: 17/913,771

(22) PCT Filed: Mar. 8, 2021
(86) PCT No.: PCT/JP2021/008996

§ 371 Date: Sep. 22, 2022

(30) Foreign Application Priority Data
Mar. 31, 2020 (JP) 2020-063877

(51) Int. Cl. G06V 20/69

(52) U.S. Cl. CPC G06V 20/693; G06V 20/695

(57) ABSTRACT

To provide a data acquisition device capable of reducing the amount of data to be output when a biological sample is imaged.

A data acquisition device including an imaging element including an acquisition unit that acquires image signals of a biological sample at two or more different time points, an information processing unit that extracts a feature amount from the image signals and generates data related to the biological sample on the basis of the feature amount, and an output control unit that causes the data related to the biological sample to be output to an outside of the imaging element, in which the signal acquisition unit, the information processing unit, and the output unit are arranged in a single chip.

### TECHNICAL FIELD

The present technology relates to a data acquisition device, a data acquisition method, and a biological sample observation system.

### BACKGROUND ART

A microscope with an incubator capable of imaging cells with an imager such as a CCD or a CMOS and monitoring the state of the cells over time is used such as when culturing cells or determining the state of reproductive cells. Furthermore, a technique for determining the state of a cell using machine learning at the time of monitoring has also been developed.

For example, Patent Document 1 describes that an evaluator that outputs an evaluation result of a state of a cell for each region of interest in a cell image, and a predictor that performs machine learning of a relationship between an evaluation result of a peripheral region of a specific region of interest in a first cell image in a pre-growth stage and a state of a cell in a specific region of interest in a second cell image at a time point after the pre-growth stage are provided, and the predictor predicts and outputs a state of a cell in a specific region of interest at a time point after a specific time point on the basis of the evaluation result of the peripheral region of a specific region of interest in a third cell image imaged at the specific time point.

Furthermore, for example, Patent Document 2 discloses an information processing device including an imaging control unit that controls an imaging mechanism so as to image a culture vessel provided with a plurality of wells accommodating cells for each imaging region, an imaging region classification unit that performs image processing on each image imaged by the imaging mechanism and classifies a plurality of the imaging regions into a first imaging region where imaging is continued and a second imaging region where imaging is not continued on the basis of an image processing result, and an observation control unit that instructs the imaging control unit to image an imaging region classified into the first imaging region and not to image an imaging region classified into the second imaging region.

For example, Patent Document 3 describes an imaging element including a pixel region in which a plurality of pixels is arranged in a matrix, and a vertical drive circuit that drives the pixels for each row, in which the vertical drive circuit includes a power supply that supplies power to an output element that outputs a drive signal for driving the pixels, and a control element that controls a current flowing between a wiring that outputs power from the power supply and a ground level according to a pulse having a predetermined pulse width at the time of switching an operation mode.

### CITATION LIST

### Patent Document

Patent Document 1: WO2018/101004
Patent Document 2: WO2018/100913
Patent Document 3: WO2018/051819

### SUMMARY OF THE INVENTION

### Problems to be Solved by the Invention

For example, in a case where a server determines the state of a cell from image data, it is necessary to transmit a large amount of image data related to an enormous number of images. When a large amount of image data related to an imaged biological sample is output, for example, restrictions or the like occur on an imaging frequency, a monitoring period, and the number of subject samples.

Accordingly, a main object of the present technology is to provide a data acquisition device capable of reducing the amount of data to be output when an image signal of a biological sample is acquired.

### Solutions to Problems

The present technology provides a data acquisition device including:
an imaging element including
a signal processing unit that acquires image signals of a biological sample at two or more different time points,
an information processing unit that extracts a feature amount from the image signals and generates data related to the biological sample on the basis of the feature amount, and
an output control unit that causes the data related to the biological sample to be output to an outside of the imaging element, in which
the signal acquisition unit, the information processing unit, and the output unit are arranged in a single chip.

The signal acquisition unit may have a configuration in which a plurality of pixels is arranged two-dimensionally, and the imaging element may be configured to image the biological sample through an objective lens.

The information processing unit may generate the data related to the biological sample using a learned model.

The information processing unit may include a feature amount extraction unit that acquires the feature amount and a state determination unit that determines a state of the biological sample on the basis of the feature amount, and the information processing unit may generate data related to a biological sample to be output on the basis of a determination result by the state determination unit.

The feature amount may be any of a feature amount related to a cell culture, a feature amount related to a fertilized egg, a feature amount related to a sperm, a feature amount related to a nucleic acid, or a feature amount related to a biological tissue piece.

The biological sample may be one kind or two or more kinds selected from a cell culture, a fertilized egg, a sperm, a nucleic acid, and a biological tissue piece.

The data related to the biological sample may include image data, alert data, flag data, or nucleic acid sequence data.

The biological sample may include a cell culture, and the information processing unit may determine whether a predetermined cell density is reached or a foreign substance is generated in the cell culture on the basis of the feature amount related to the cell culture.

The biological sample may include a cell culture, and the information processing unit may generate image data of the cultured cell on the basis of the feature amount related to the cell culture.

The biological sample may include a fertilized egg, and the information processing unit may determine whether a predetermined division process has been reached on the basis of the feature amount related to the fertilized egg.

The biological sample may include a fertilized egg, and the information processing unit may generate image data of the fertilized egg on the basis of a feature amount related to the fertilized egg.

The biological sample may include sperm, and the information processing unit may determine a state of the sperm on the basis of the feature amount related to the sperm.

The biological sample may include sperm, and the information processing unit may generate image data of the sperm on the basis of the feature amount related to the sperm.

The biological sample may include a nucleic acid, and the information processing unit may generate sequence data of the nucleic acid on the basis of the feature amount related to the nucleic acid.

The present technology also provides a data acquisition method including:
a feature amount extraction step of extracting a feature amount from image data obtained by imaging a biological sample by an imaging element at two or more different time points;
a data generation step of generating data related to the biological sample on the basis of the feature amount; and
an output step of causing the data related to the biological sample to be output to an outside of the imaging element.

The present technology provides a biological sample observation system including:
a holding unit capable of holding a biological sample;
an irradiation unit that irradiates the biological sample with light; and
an imaging element including
a signal acquisition unit that acquires image signals of the biological sample at two or more different time points,
an information processing unit that extracts a feature amount from the image signals and generates data related to the biological sample on the basis of the feature amount, and
an output control unit that causes the data related to the biological sample to be output to an outside of the imaging element, in which
the signal acquisition unit, the information processing unit, and the output unit are arranged in a single chip.

An incubator that stores the holding unit may be further included.

The biological sample observation system may be a microscopic observation system.

The biological sample observation system may be a nucleic acid sequence analysis system.

### BRIEF DESCRIPTION OF DRAWINGS

FIG. 1 is a block diagram illustrating a configuration example in a data acquisition device according to the present technology.

FIG. 2 is a block diagram illustrating a configuration example of an imaging device 2.

FIG. 3 is a perspective view illustrating an outline of an external configuration example of the imaging device 2.

FIG. 4 is a schematic diagram illustrating a configuration example of a system according to the present technology.

FIG. 5 is an example of a flow of processing by the data acquisition device according to the present technology.

FIG. 6 is an example of a flow of processing by the data acquisition device according to the present technology.

FIG. 7 is a block diagram schematically illustrating a processing procedure example of a general specialized AI type.

FIG. 8 is an example of a flow of processing by the data acquisition device according to the present technology.

FIG. 9 is an example of a flow of processing by the data acquisition device according to the present technology.

FIG. 10 is an example of data processing related to a cell culture according to the present technology.

FIG. 11 is an example of data processing related to a fertilized egg according to the present technology.

FIG. 12 is an example of data processing related to sperm according to the present technology.

FIG. 13 is an example of data processing related to nucleic acids according to the present technology.

FIG. 14 is an example of a flow of data processing related to nucleic acids according to the present technology.

FIG. 15 is an example of data processing related to a biological tissue piece according to the present technology.

### MODE FOR CARRYING OUT THE INVENTION

Hereinafter, preferred embodiments for carrying out the present technology will be described. Note that the embodiments described below are representative embodiments of the present technology, and the scope of the present technology is not limited only to these embodiments. Note that the present technology will be described in the following order.
1. First Embodiment (Data Acquisition Device)
(1) Description of First Embodiment
(1-1) Imaging Element
(1-2) Signal Acquisition Unit
(1-3) Imaging Processing Unit
(1-4) Information Processing Unit
(1-5) Output Control Unit
(1-6) Output Unit and Input Unit
(1-7) Illumination Optical System
(1-8) Observation Optical System
(2) Configuration Example of Imaging Element
(3) First Example of First Embodiment
(4) Example of Processing of Data by Imaging Element in First Embodiment
(4-1) First Example of Processing of Data by Imaging Element in First Embodiment
(4-2) Second Example of Processing of Data by Imaging Element in First Embodiment
(4-3) Third Example of Processing of Data Related to Cell Culture by Imaging Element
(4-4) Fourth Example of Processing of Data Related to Fertilized Egg by Imaging Element
(4-5) Fifth Example of Processing of Data Related to Sperm by Imaging Element
(4-6) Sixth Example of Processing of Data Related to Nucleic Acid by Imaging Element
(4-7) Seventh Example of Processing of Data related to Biological Tissue Piece by Imaging Element
2. Second Embodiment (Application Device)
3. Third Embodiment (Data Acquisition Method)
4. Fourth Embodiment (Program)
5. Fifth Embodiment (Biological Sample Observation System)

### 1. First Embodiment (Data Acquisition Device)

### (1) Description of First Embodiment

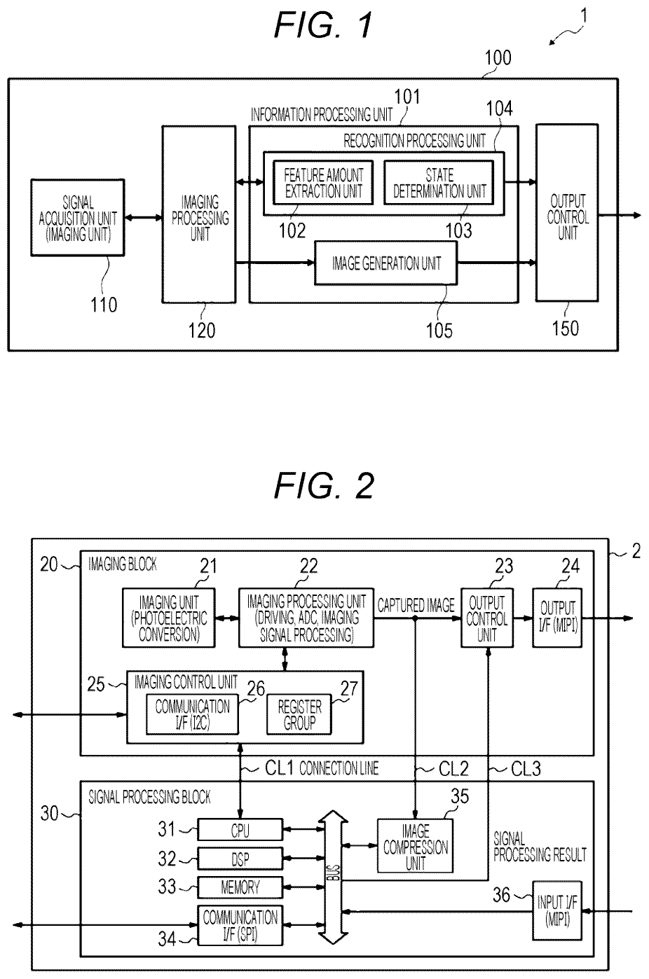

An example of a data acquisition device 1 according to the present technology will be described with reference to FIG. 1. However, the present technology is not limited to this description.

The data acquisition device 1 according to the present technology includes an imaging element 100. The imaging element 100 includes a signal acquisition unit 110, an imaging processing unit 120, an information processing unit 101, and an output control unit 150.

The data acquisition device 1 may further include an illumination optical system, an observation optical system, a nucleic acid sequence analysis system, and the like. The data processing device 1 may be provided in, for example, a biological sample observation system or the like, and examples of the biological sample observation system include, but are not limited to, a microscopic observation system, a nucleic acid sequence analysis system, and the like.

The data acquisition device 1 may further include a memory that temporarily stores data related to the biological sample output from the imaging element 100, image data, and the like.

### (1-1) Imaging Element

The imaging element 100 includes a signal acquisition unit 110 that acquires image signals of the biological sample at two or more different time points, and an information processing unit 101 that extracts a feature amount from the image signals and generates the data related to the biological sample on the basis of the feature amount.

Moreover, the imaging element 100 may include an output control unit 150 that causes the data related to the biological sample to be output to the outside of the imaging element. Thus, the imaging element 100 can reduce the amount of data to be output to the outside of the imaging element when outputting data related to the biological sample including image data and the like. Further, since the amount of data to be output can be reduced, the imaging element 100 is also suitable for, for example, long-time observation, real-time observation, increasing the number of observation subjects, and the like. Furthermore, since the imaging element 100 can reduce the amount of data to be output, a load of data transfer can be reduced, and processing speed can be improved.

The image data obtained by the imaging element 100 may be, for example, moving image data or time-lapse image data.

The imaging element 100 is configured to generate the data related to the biological sample on the basis of the acquired image signal, and output the generated the data related to the biological sample to the outside of the imaging element (for example, a server or a device) via the output control unit 150. Thus, it is not necessary to continuously or temporally output the acquired image signal with the amount of data, so that the amount of data to be output can be reduced.

Moreover, the amount of data to be output as described above can be reduced by the imaging element 100. Thus, the imaging interval can be further shortened, so that the state regarding the biological sample can be determined with higher accuracy. Moreover, it is also possible to monitor the biological sample for a long time. In addition, many biological samples can be monitored collectively.

Furthermore, the imaging element 100 can control output timing of acquired image data on the basis of the determination result of the state related to the biological sample. The imaging element 100 can also compress and output the amount of data to be output. Furthermore, the imaging element 100 can also generate and output image data obtained by imaging at a short imaging interval only when it is an important state for the biological sample (for example, drug administration or division process of fertilized egg).

Moreover, the imaging element 100 may further include a signal acquisition unit 110 that images the biological sample, and an imaging processing unit 120 that controls imaging of the imaging unit.

The imaging element 100 may be configured to image the biological sample via an objective lens. Note that the device including the objective lens may be either an upright type or an inverted type.

Preferably, the imaging element 100 includes a signal acquisition unit in which a plurality of pixels is two-dimensionally arranged, and the signal acquisition unit 110 and the information processing unit 101 are arranged in one chip. The imaging element 100 is preferably, for example, a complementary metal oxide semiconductor (CMOS) image sensor formed by one chip. The imaging element 100 is preferably configured to receive incident light from a light source, perform photoelectric conversion, and be capable of outputting an image signal corresponding to the incident light from the light source. Note that the light of the light source may be either natural light or artificial light.

### (1-2) Signal Acquisition Unit

The signal acquisition unit 110 acquires image signals of the biological sample at two or more different time points. The signal acquisition unit 110 may be configured by arranging a plurality of pixels two-dimensionally. The signal acquisition unit 110 may acquire the image signals by imaging, for example, and in this case, the signal acquisition unit 110 may also be referred to as an imaging unit. The signal acquisition unit 110 can be driven by the imaging processing unit 120, image the biological sample, and acquire an image signal. The signal acquisition unit 110 can acquire image signals of the biological sample at two or more different time points. For example, light from the biological sample enters the signal acquisition unit 110. The signal acquisition unit 110 receives incident light from the biological sample in each pixel, performs photoelectric conversion, and outputs an analog image signal corresponding to the incident light.

Note that the size of the image (signal) output by the signal acquisition unit 110 can be selected from, for example, a plurality of sizes, such as 12 M (3968×2976) pixels and a video graphics array (VGA) size (640×480 pixels).

Furthermore, for the image output by the signal acquisition unit 110, for example, it is possible to select whether to use a color image of Red, Green, and Blue (RGB) or a monochrome image of only luminance.

These selections can be made as a type of imaging mode setting.

### (1-3) Imaging Processing Unit

The imaging processing unit 120 can perform control of imaging processing related to imaging at the signal acquisition unit

110, such as driving of the signal acquisition unit 110, analog to digital (AD) conversion of an analog image signal output from the signal acquisition unit 110, and imaging signal processing, for example.

The analog image signal output from the signal acquisition unit 110 is converted into a digital image signal by AD conversion by the imaging processing unit 120.

Here, examples of the imaging signal processing include processing of obtaining brightness for each small region by calculating an average value of pixel values for each predetermined small region with respect to the image signal output by the signal acquisition unit 110, or the like, processing of converting the image signal output from the signal acquisition unit 110 into a high dynamic range (HDR) image, defect correction, development, and the like.

Furthermore, the imaging processing unit 120 may control the signal acquisition unit 110 in accordance with imaging information regarding imaging and other various types of information.

The imaging information and the like are not particularly limited, but more specifically, for example, ISO sensitivity (analog gain at the time of AD conversion in imaging processing), exposure time (shutter speed), frame rate, focus, imaging mode, (information indicating) a cutout range and the like, and the like can be employed. The imaging mode may include, for example, a manual mode in which the exposure time, the frame rate, and the like are manually set, and an automatic mode in which the exposure time, the frame rate, and the like are automatically set according to the scene. For example, the automatic mode may include a mode corresponding to various imaging scenes such as a type of the observation subject, a state of the observation subject, and an observation situation.

### (1-4) Information Processing Unit

The information processing unit 101 includes a recognition processing unit 104 including a feature amount extraction unit 102 that extracts and acquires a feature amount from an image signal acquired by imaging a biological sample at two or more different time points, and a state determination unit 103 that determines a state of the biological sample on the basis of the feature amount.

Examples of the biological sample include a cell culture, a fertilized egg, a sperm, nucleic acids, a biological tissue piece, and the like, and one kind or two or more kinds can be selected from them.

Examples of the feature amount include a feature amount related to a cell culture, a feature amount related to a fertilized egg, a feature amount related to a sperm, a feature amount related to nucleic acids, a feature amount related to a biological tissue piece, or the like, and one kind or two or more kinds can be selected from them.

Preferably, the information processing unit 101 is configured to generate the data related to the biological sample on the basis of a determination result by the state determination unit 103.

Examples of the data related to the biological sample include image data, alert data, flag data, and nucleic acid sequence data, attention data, and the like. The information processing unit 101 can select one kind or two or more kinds selected from them as the data related to the biological sample.

The information processing unit 101 preferably generates the data related to the biological sample using the learned model.

The information processing unit 101 may include an image generation unit 105 configured to generate the data related to the biological sample to be output from the acquired image signal on the basis of the determination result by the state determination unit 103. The image signal may be data obtained by imaging by the signal acquisition unit 110 and subjected to the imaging processing unit 120.

The data related to the biological sample generated by the information processing unit 101 may include, for example, one kind or two or more kinds selected from image data, alert data, flag data, nucleic acid sequence data, and attention data. In a case where the data related to the biological sample is a combination of two or more kinds of these data, one data may be associated with the other data.

The image generation unit 105 can receive the image signal related to the biological sample from the signal acquisition unit 110 via the imaging processing unit 120. The image generation unit 105 can generate, for example, image data on the basis of the received image signal. The image generation unit 105 may directly transmit the image data as it is to the output control unit 150, or the image generation unit 105 may compress the image data related to the biological sample and transmit the obtained compressed image data to the output control unit 150.

The recognition processing unit 104 can include, for example, a feature amount extraction unit that extracts a feature amount from image signals of the biological sample at two or more different time points, and a state determination unit that determines a state of the biological sample on the basis of the feature amount. The information processing unit 101, particularly the recognition processing unit 104 can generate the data related to the biological sample to be output on the basis of the determination result by the state determination unit.

In this manner, the imaging element generates the data related to the biological sample in the imaging element. For example, by outputting the data related to the biological sample other than the image data without outputting the image data, the amount of data to be output from the imaging element can be reduced.

Furthermore, the recognition processing unit 104 determines image data whose compression rate is changed according to the priority (for example, presence or absence of flag data) of the image data related to the biological sample from the plurality of acquired image signals on the basis of the determination result by the state determination unit 103. On the basis of this determination result, the image generation unit 105 can perform compression processing on the image data and generate the image data as the data related to the biological sample. For example, in a case where it is determined that the priority is high (for example, there is flag data), the image generation unit 105 may output uncompressed image data or image data compressed at a lower compression rate (for example, image data with higher resolution or the like) to the outside of the imaging element via the output control unit 150. Furthermore, for example, in a case where it is determined that the priority is low (for example, no flag data), the image generation unit 105 does not have to output the image data to the outside of the imaging element, or may output image data with a higher compression rate or alert data (for example, character data or the like) instead of the image data to the outside of the imaging element via the output control unit 150. Furthermore, in a case of signal data (for example, alert data or the like) or the like that does not require generation of image data, the recognition processing unit 104 may output the signal data (for example, alert data or the like) or the like to the outside of the imaging element via the output control unit 150. Thus, the amount of data to be output to the outside of the imaging element can be reduced.

Furthermore, the recognition processing unit 104 may select a region of an image to be output to the outside of the imaging element on the basis of a determination result by the state determination unit 103 in the imaged image. On the basis of the determination result, the image generation unit 105 can perform compression processing on the imaged image into image data of only this region or image data including only this region and its peripheral pixels, and can generate the image data as the data related to the biological sample. For example, examples of the compression processing include generating image data of only a necessary region, and generating image data from which a region other than this region is removed, and the like. Note that the data related to the biological sample may include coordinate (for example, an x axis, a y axis, a z axis, a t (time) axis, or the like) position data of the region and image data associated with the coordinate position data. Thus, the amount of data to be output to the outside of the imaging element can be reduced.

Furthermore, the recognition processing unit 104 generates nucleic acid sequence data of nucleic acid in a spot that emits a signal in an imaged image on the basis of the determination result by the state determination unit 103. Examples of criteria for determination by the state determination unit 103 include data related to a spot, for example, optical characteristics, a fluorescence wavelength, a fluorescence spectrum, an absorption spectrum, an area, luminance, a distance from the center, and extraction of a circular shape (for example, hough conversion), and the like. Furthermore, the type of nucleic acids can also be determined from the data related to the spot (for example, optical characteristics, fluorescence wavelength, fluorescence spectrum, absorption spectrum, and the like). More specifically, the determination of the type of nucleic acids may be performed by analyzing a characteristic of a signal labeled in the nucleic acid. The characteristic analysis can be performed, for example, by measuring a characteristic such as a fluorescence wavelength of a fluorescent dye by a filter method or a spectral method. Furthermore, the number of nucleic acids can also be determined from optical characteristics, a fluorescence wavelength, a fluorescence spectrum, an absorption spectrum, an area, luminance, a distance from the center, and the like regarding the spot. As described above, the recognition processing unit 104 can determine, for example, the type and/or number of nucleic acids on the basis of the data related to the spot. The recognition processing unit 104 can generate nucleic acid sequence data on the basis of the determined type and/or number of nucleic acids.

In addition, on the basis of the determination result by the state determination unit 103, the image generation unit 105 may regularly divide (for example, it is divided into grids and blocks) an image of image data, set a coordinate position of a spot that emits a signal, associate the coordinate position with the nucleic acid sequence data of each spot, and include the coordinate position in the data related to the biological sample. That is, the image generation unit 105 can generate data related to the biological sample including coordinate position data of each spot and nucleic acid sequence data associated with the coordinate position data. Furthermore, on the basis of the determination result by the state determination unit 103, the image generation unit 105 can also perform compression processing on the image data by excluding the image data related to a region other than the spot. Thus, the amount of data to be output to the outside of the imaging element can be reduced.

In addition, from the image of the image data obtained by imaging, on the basis of the determination result by the state determination unit 103, the image generation unit 105 can generate the data related to the biological sample so as to detect a feature region and add a flag to the region. Furthermore, the image generation unit 105 can also generate the data related to the biological sample so as to detect the image data including the feature region from the image data group obtained by imaging on the basis of the determination result and add a flag to the image data. Note that the determination at this time can take into consideration the length of the observation time, the slow moving speed, and the like.

### (1-5) Output Control Unit

The output control unit 150 is configured to cause the data related to the biological sample to be output to the outside of the imaging element. The output control unit 150 may cause the image data to be output. Preferably, the output control unit 150 can control, for example, whether to cause the imaging element 100 to output data related to the biological sample including the image data or output data related to the biological sample not including the image data. By the control, for example, the data related to the biological sample including the image data can be output as necessary, and in other cases, the data related to the biological sample not including the image data can be output. Thus, data to be output from the imaging element can be reduced. Furthermore, the output control unit 150 may cause the imaging element 100 to output alert data or the like generated by the information processing unit 101.

### (1-6) Output Unit and Input Unit

The data acquisition device 1 may include an output unit. The output unit can output data and/or image data related to the biological sample that has been output from the imaging element. Moreover, the output unit may output an alert on the basis of the alert data. The output unit may include, for example, a display device that displays an image. Furthermore, the output unit may include a speaker or the like that outputs sound.

The data acquisition device 1 may include an input unit. The input unit receives a user operation. The input unit may include, for example, a mouse and/or a keyboard or the like. Furthermore, a display surface of the display device may be configured as an input unit that receives a touch operation.

The data acquisition device 1 may include a storage unit. The storage unit can store data and/or image data related to the biological sample that has been output from the imaging element. Furthermore, the storage unit may store alert data. The storage unit may include, for example, a recording medium.

### (1-7) Illumination Optical System

The irradiation optical system is an optical system for illuminating the subject S during imaging by the imaging element 100. The irradiation optical system includes a light source for illumination, and can irradiate the subject S with visible light or ultraviolet light, for example. The light source included in the irradiation optical system may be appropriately selected by those skilled in the art according to the type of image data to be acquired by the imaging element 100, and may include, for example, at least one selected from a halogen lamp, an LED lamp, a mercury lamp, and a xenon lamp. For example, in a case where the image data is bright field image data, the irradiation optical system may include, for example, an LED lamp or a halogen lamp. In a case where the image data is fluorescence image data, the irradiation optical system may include, for example, an LED lamp, a mercury lamp, or a xenon lamp. Depending on the type of phosphor emitting the fluorescence, the wavelength of the emitted light or the type of lamp may be selected.

### (1-8) Observation Optical System

The observation optical system is configured to enable the imaging element 100 to enlarge and image the subject S. The observation optical system may include, for example, an objective lens. Furthermore, the observation optical system may include a relay lens for relaying the image enlarged by the objective lens to the imaging element 100. The configuration of the observation optical system may be selected according to the subject S. For example, the magnification of the objective lens can be appropriately selected according to, for example, the subject S. Furthermore, the configuration of the relay lens can be appropriately selected according to, for example, the objective lens and the imaging element 100. The observation optical system may include an optical component other than the objective lens and the relay lens.

### (2) Configuration Example of Imaging Element

Hereinafter, a more specific configuration example of the imaging element 100 will be described in detail with reference to FIG. 2, but the configuration of the imaging element is not limited to this example.

As illustrated in FIG. 2, the imaging element 100 includes an imaging block 20 and a signal processing block 30. The imaging block 20 and the signal processing block 30 are electrically connected by connection lines (internal buses) CL1, CL2, and CL3.

The imaging block 20 includes an imaging unit 21, an imaging processing unit 22, an output control unit 23, an output I/F 24, and an imaging control unit 25.

The signal processing block 30 may include a central processing unit (CPU) 31, a digital signal processor (DSP) 32, and a memory 33. The signal processing block 30 may further include a communication I/F 34, an image compression unit 35, and an input I/F 36. The signal processing block 30 performs predetermined signal processing using the entire image data obtained by the imaging unit. The processing (for example, processing of extracting the feature amount and processing of generating the data related to the biological sample) by the information processing unit 101 described above is implemented by the signal processing block 30.

These components of the imaging element 100 will be described below.

The imaging unit 21 corresponds to the signal acquisition unit 110 described above in “(1-2) Signal acquisition unit”. The imaging unit 21 images the entire subject S including the living tissue. The imaging unit 21 can be driven by, for example, the imaging processing unit 22 to perform the imaging. The imaging unit 21 may include, for example, a plurality of pixels arranged in a two-dimensional manner. Each pixel included in the imaging unit 21 receives light, performs photoelectric conversion, and then outputs an analog image signal based on the received light.

The size of the image (signal) output by the imaging unit 21 can be selected from a plurality of sizes such as 12 M (3968×2976) pixels or a video graphics array (VGA) size (640×480 pixels). The image output by the imaging unit 21 may be a color image or a monochrome image. The color image can be represented by, for example, RGB (Red, Green, Blue). The monochrome image can be represented by, for example, luminance. These selections can be made as a type of imaging mode setting.

The imaging processing unit 22 can perform imaging processing related to imaging of an image by the imaging unit 21. For example, under the control of the imaging control unit 25, the imaging processing unit 22 can perform imaging processing such as
driving of the imaging unit 21, analog to digital (AD) conversion of an analog image signal output from the imaging unit 21, imaging signal processing, or the like.

More specifically, the imaging signal processing can be, for example, processing of obtaining brightness for each of predetermined small regions by calculating an average value of pixel values for each of the small regions, or the like for an image output from the imaging unit 21, processing of converting the image output from the imaging unit 21 into a high dynamic range (HDR) image, defect correction, or development.

The imaging processing unit 22 can output a digital image signal (for example, an image of 12 M pixels or VGA size) obtained by AD conversion or the like of the analog image signal output from the imaging unit 21 as an imaged image.

The imaged image output from the imaging processing unit 22 can be supplied to the output control unit 23. Furthermore, the imaged image output by the imaging processing unit 22 can be supplied to the signal processing block 30 (in particular, the image compression unit 35) via the connection line CL2.

The imaged image can be supplied from the imaging processing unit 22 to the output control unit 23. Furthermore, a determination result using, for example, an imaged image or the like can be supplied from the signal processing block 30 to the output control unit 23 via the connection line CL3.

The output control unit 23 performs output control of selectively outputting the imaged image supplied from the imaging processing unit 22 and the determination result by the signal processing block 30 from the (one) output I/F 24 to the outside of the imaging element 100.

That is, the output control unit 23 selects the imaged image from the imaging processing unit 22 or the determination result from the signal processing block 30, and supplies this imaged image or determination result to the output I/F 24.

The output I/F 24 is an I/F that outputs the imaged image and the determination result supplied from the output control unit 23 to the outside. As the output I/F 24, for example, a relatively high-speed parallel I/F such as a mobile industry processor interface (MIPI) can be employed. The output I/F 24 outputs the imaged image from the imaging processing unit 22 or the determination result from the signal processing block 30 to the outside according to the output control by the output control unit 23. Therefore, for example, in a case where only the determination result from the signal processing block 30 is necessary on the outside and the imaged image itself is not necessary, only the determination result can be output, and the amount of data to be output from the output I/F 24 to the outside can be reduced.

Furthermore, the signal processing block 30 performs determination processing to obtain a determination result used by a component external to the imaging element 100 (for example, a second imaging element 112 and/or a control unit 113 (not illustrated)), and the determination result is output from the output I/F 24. Thus, it is not necessary to perform signal processing on the outside, and a load on an external block can be reduced.

The imaging control unit 25 can control the imaging processing unit 22 according to the imaging information (image data or the like) stored in the register group 27, thereby controlling imaging by the imaging unit 21.

The register group 27 can store imaging information, a result of imaging signal processing in the imaging processing unit 22, and output control information regarding output control in the output control unit 23. The output control unit 23 can perform output control of selectively outputting the imaged image (imaged image data or the like) and the determination result according to the output control information stored in the register group 27.

The imaging control unit 25 and the CPU included in the signal processing block 30 may be connected via the connection line CL1. The CPU can read and write information from and to the register group 27 via the connection line. That is, reading and writing of information from and to the register group 27 may be performed by the communication I/F 26 or may be performed by the CPU.

The signal processing block 30 determines a feature related to the subject on the basis of the entire image data. The signal processing block 30 may include, for example, a central processing unit (CPU) 31, a digital signal processor (DSP) 32, and a memory 33. The signal processing block 30 may further include a communication I/F 34, an image compression unit 35, and an input I/F 36. The determination unit 30 can perform predetermined signal processing using the entire image data obtained by the imaging unit.

The CPU 31, the DSP 32, the memory 33, the communication I/F 34, and the input I/F 36 constituting the signal processing block 30 are connected to each other via a bus, and can exchange information as necessary.

The CPU 31 executes the program stored in the memory 33 to perform various processes such as control of the signal processing block 30 or reading and writing of information from and to the register group 27 of the imaging control unit 25, for example. For example, by executing the program, the CPU 31 functions as an imaging information calculation unit that calculates imaging information by using a signal processing result obtained by signal processing in the DSP 32, and can feed back new imaging information calculated by using the signal processing result to the register group 27 of the imaging control unit 25 via the connection line CL1 and cause the new imaging information to be stored therein. Therefore, the CPU 31 can control imaging by the imaging unit 21 and/or imaging signal processing by the imaging processing unit 22 according to the signal processing result of the imaged image. Furthermore, the imaging information stored in the register group 27 by the CPU 31 can be provided (output) to the outside from the communication I/F 26. For example, the focus information in the imaging information stored in the register group 27 can be provided from the communication I/F 26 to a focus driver (not illustrated) that controls the focus.

By executing the program stored in the memory 33, the DSP 32 functions as a signal processing unit that performs signal processing using an imaged image supplied from the imaging processing unit 22 to the signal processing block 30 via the connection line CL2 and information received by the input I/F 36 from the outside.

The memory 33 may include a static random access memory (SRAM), a dynamic RAM (DRAM), or the like. The memory 33 stores various data such as data used for processing of the signal processing block 30, for example.

For example, the memory 33 stores a program received from the outside via the communication I/F 34, an imaged image compressed by the image compression unit 35, particularly, an imaged image used in the signal processing in the DSP 32, the signal processing result of the signal processing performed in the DSP 32, information received by the input I/F 36, or the like.

The communication I/F 34 is, for example, a second communication I/F such as a serial communication I/F such as a serial peripheral interface (SPI), and exchanges necessary information such as a program executed by the CPU 31 or the DSP 32 with an external component (for example, a memory of the outside of the first imaging element 111, an information processing device, or the like).

For example, the communication I/F 34 downloads a program executed by the CPU 31 or the DSP 32 from the outside, supplies the program to the memory 33 and causes the program to be stored therein. Therefore, various processes can be executed by the CPU 31 or the DSP 32 by the program downloaded by the communication I/F 34. Note that the communication I/F 34 can exchange not only programs but also arbitrary data with the outside. For example, the communication I/F 34 can output the signal processing result obtained by the signal processing in the DSP 32 to the outside. Furthermore, the communication I/F 34 outputs information according to an instruction of the CPU 31 to an external device, so that the external device can be controlled according to the instruction of the CPU 31.

Here, the signal processing result obtained by the signal processing in the DSP 32, in addition to being output from the communication I/F 34 to the outside, can be written in the register group 27 of the imaging control unit 25 by the CPU 31. The signal processing result written in the register group 27 can be output from the communication I/F 26 to the outside. This similarly applies to the processing result of the processing performed by the CPU 31.

An imaged image is supplied from the imaging processing unit 22 to the image compression unit 35 via the connection line CL2. The image compression unit 35 performs compression processing for compressing the imaged image, and generates a compressed image having a smaller amount of data than the imaged image.

The compressed image generated by the image compression unit 35 is supplied to the memory 33 via the bus and stored therein.

Here, the signal processing in the DSP 32 can be performed using not only the imaged image itself but also the compressed image generated from the imaged image by the image compression unit 35. Since the compressed image has a smaller amount of data than the imaged image, it is possible to reduce the load of the signal processing in the DSP 32 and to save the storage capacity of the memory 33 that stores the compressed image.

As the compression processing in the image compression unit 35, for example, scale-down for converting an imaged image of 12 M (3968×2976) pixels into an image of a VGA size can be performed. Furthermore, in a case where the signal processing in the DSP 32 is performed on luminance and the imaged image is an RGB image, YUV conversion for converting the RGB image into, for example, a YUV image can be performed as the compression processing.

Note that the image compression unit 35 can be implemented by software or can be implemented by dedicated hardware.

The input I/F 36 is an I/F that receives information from the outside. The input I/F 36 receives, for example, an output of an external sensor (external sensor output) from the external sensor, and supplies the output to the memory 33 via the bus and causes the data to be stored therein.

For example, similarly to the output I/F 24, a parallel I/F such as a mobile industry processor interface (MIPI) can be employed as the input I/F 36.

Furthermore, as the external sensor, for example, a distance sensor that senses information regarding distance can be employed, and moreover, as the external sensor, for example, an image sensor that senses light and outputs an image corresponding to the light, that is, an image sensor different from an imaging device 2 can be employed.

In the DSP 32, besides using (the compressed image generated from) the imaged image, the signal processing can be performed using the external sensor output received by the input I/F 36 from the external sensor as described above and stored in the memory 33.

In the one-chip imaging element 100 configured as described above, signal processing using (a compressed image generated from) an imaged image obtained by imaging by the imaging unit 21 is performed by the DSP 32, and a signal processing result of the signal processing and the imaged image are selectively output from the output I/F 24. Therefore, it is possible to downsize the imaging device that outputs information needed by the user.

Here, in a case where the signal processing of the DSP 32 is not performed in the imaging element 100, and thus the signal processing result is not output from the imaging element 100 and an imaged image is output, that is, a case where the imaging element 100 is configured as an image sensor that merely captures and outputs an image, the imaging element 100 can be configured with only the imaging block 20 which is not provided with the output control unit 23.

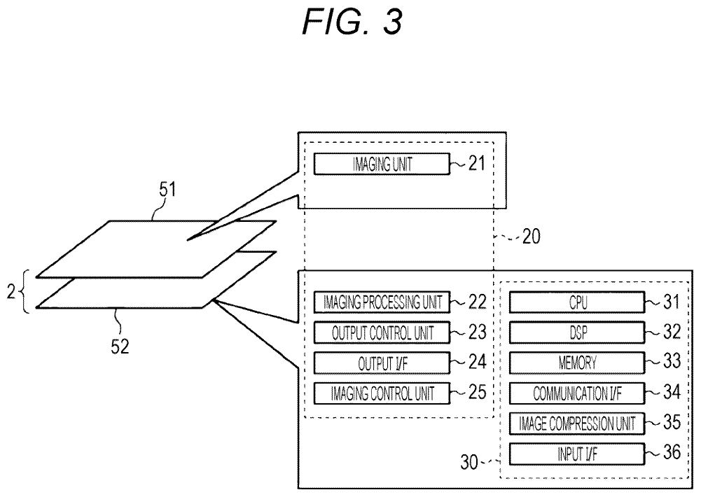

FIG. 3 is a perspective view illustrating an outline of an external configuration example of the imaging element 100 in FIG. 1.

For example, as illustrated in FIG. 3, the imaging element 100 can be configured as a one-chip semiconductor device having a stacked structure in which a plurality of dies is stacked.

In FIG. 3, the imaging element 100 is configured by stacking two dies of dies 51 and 52.

In FIG. 3, the imaging unit 21 is mounted on the upper die 51, and the imaging processing unit 22 to the imaging control unit 25 and the CPU 31 to the input I/F 36 are mounted on the lower die 52.

The upper die 51 and the lower die 52 are electrically connected by, for example, forming a through hole that penetrates the die 51 and reaches the die 52, or performing Cu—Cu bonding for directly connecting a Cu wiring exposed on a lower surface side of the die 51 and a Cu wiring exposed on an upper surface side of the die 52, or the like.

Here, in the imaging processing unit 22, as a method of performing AD conversion of the image signal output from the imaging unit 21, for example, a column-parallel AD method or an area AD method can be employed.

In the column-parallel AD method, for example, an AD converter (ADC) is provided for a column of pixels constituting the imaging unit 21, and the ADC in each column is in charge of AD conversion of a pixel signal of a pixel in the column, so that AD conversion of an image signal of a pixel in each column in one row is performed in parallel. In a case where the column-parallel AD method is employed, a part of the imaging processing unit 22 that performs AD conversion of the column-parallel AD method may be mounted on the upper die 51.

In the area AD method, pixels constituting the imaging unit 21 are divided into a plurality of blocks, and an ADC is provided for each block. Then, the ADC of each block is in charge of AD conversion of the pixel signals of the pixels of the block, so that AD conversion of image signals of the pixels of the plurality of blocks is performed in parallel. In the area AD method, AD conversion (reading and AD conversion) of image signals can be performed only for necessary pixels among the pixels constituting the imaging unit 21 with a block as a minimum unit.

Note that, if the area of the imaging element 100 is allowed to be large, the imaging element 100 can be configured with one die.

Furthermore, in FIG. 3, the imaging element 100 of one chip is configured by stacking the two dies 51 and 52, but the imaging element 100 of one chip can be configured by stacking three or more dies. For example, in a case where the imaging element 100 of one chip is configured by stacking three dies, the memory 33 of FIG. 3 can be mounted on another die.

In a case where the information needed by the user is an imaged image, the imaging element 100 can output the imaged image.

Furthermore, in a case where the information needed by the user is obtained by signal processing using an imaged image, the imaging element 100 can obtain and output a signal processing result as the information needed by the user by performing this signal processing in the DSP 32.

### (3) First Example of First Embodiment

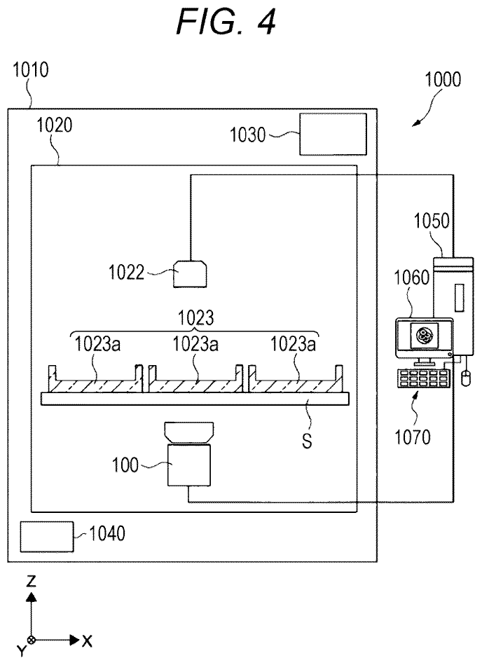

The data acquisition device according to the present technology may be configured as, for example, a device provided with an imaging element that processes and outputs image data obtained by imaging a biological sample at two or more different time points. An example of the data acquisition device according to the present technology configured as described above and an example of processing by the data processing device will be described below with reference to FIG. 4. However, the present technology is not limited to this description.

FIG. 4 illustrates a biological sample observation system 1000 including the data processing device 1 provided with the imaging element 100 according to the present technology, but the present technology is not limited to the biological sample observation system. The biological sample observation system 1000 is configured as a system for observing a biological sample, and may be configured as a system for performing cell culture, cell recovery, fluorescence reaction, and the like.

As the biological sample in the system for observing the biological sample, for example, one kind or two or more kinds selected from a cell culture, a fertilized egg, a sperm, nucleic acids, and a biological tissue piece can be used, but the biological sample is not particularly limited thereto.

Examples of a system for observing a biological sample such as a cell culture, a fertilized egg, a sperm, and a biological tissue piece include a culture system and a microscopic observation system. Furthermore, examples of a system for observing a biological sample such as nucleic acids include a nucleic acid sequence analysis system.

The biological sample observation system 1000 may include, for example, a holding unit capable of holding a biological sample, and an irradiation unit that irradiates the biological sample with light. The biological sample observation system 1000 may further include an incubator that stores the holding unit.

The description regarding the illumination optical system described in “(1-7) Illumination optical system” described above applies to the irradiation unit.

The holding unit may include a container or a plate in which one or a plurality of biological samples can be accommodated or mounted, and the like. The container, the plate, or the like may be used for observation and/or culture of a biological sample. Examples of the container, the plate, and the like include, but are not limited to, a well, an assay plate, a microplate, a microscope slide, and the like.

For example, FIG. 4 illustrates an example of a system for observing a cell culture, a fertilized egg, a sperm, and the like.

As illustrated in FIG. 4, the biological sample observation system 1000 can include an incubator 1010, an observation device 1020, a humidity-temperature-gas control unit 1030, a detection unit 1040, the data acquisition device 1 provided with the imaging element 100, a personal computer (PC) 1050, an output unit 1060, and an input unit 1070.

The incubator 1010 is a culture apparatus capable of accommodating the observation device 1020, the humidity-temperature-gas control unit 1030, and the detection unit 1040, and may have a function of keeping the internal temperature, humidity, and the like constant. The incubator 1010 may be configured to allow any gas to flow in. The kind of the gas is not particularly limited, and is, for example, one kind or two or more kinds selected from nitrogen, oxygen, carbon dioxide, and the like.

The observation device 1020 includes the data acquisition device 1 provided with the imaging element 100, a light source 1022, and a container group 1023 that stores a biological sample. The light source 1022 can function as an irradiation unit that irradiates the biological sample with light. The container group 1023 accommodating the biological sample can function as a holding unit capable of holding the biological sample. The imaging element 100 includes the signal acquisition unit 110 for imaging the biological sample.

The imaging element 100 can image the biological sample accommodated in the container 1023a (desh) that accommodated the biological sample over time. Although the imaging element 100 is arranged in a downward direction with respect to the biological sample in FIG. 4, the arrangement is not particularly limited, and may be arranged in any direction such as a vertical direction, a front-back direction, and a left-right direction. The observation device may be either an upright type or an inverted type. The imaging direction in the imaging element 100 may be any of XYZ directions, and is not particularly limited. The imaging element 100 may be configured to be movable in an optical axis direction (Z-axis direction) and a horizontal direction (a direction orthogonal to the Z-axis direction) for imaging. Further, the imaging element 100 may be configured to image the biological sample via the objective lens.

Furthermore, the data acquisition device 1 may be configured to be capable of imaging a still image or a moving image.

The light source 1022 is not particularly limited, and for example, a light emitting diode (LED) capable of emitting light having a specific wavelength, a visible light lamp, a xenon lamp, or the like can be employed.

The container group 1023 may include a plurality of containers. The arrangement of the container group 1023 is not particularly limited, and for example, the container group 1023 can be arranged on an observation stage S between the imaging element 100 and the light source 1022, and at this time, the observation stage S can be configured to be capable of transmitting light emitted by the light source 1022.

In addition, the material constituting the container group 1023 is not particularly limited, and is preferably a material that can transmit irradiated light.

The humidity-temperature-gas control unit 1030 controls the temperature and humidity in the incubator 1010 and the gas induced in the incubator 1010, and can control the temperature to, for example, about 37 to 38° C. suitable for cell culture.

The detection unit 1040 can be configured to detect a temperature, humidity, and atmospheric pressure in the incubator 1010, illuminance of the light source 1022, and the like, and output them to the data acquisition device 1.

The data acquisition device 1 is as described above in “(1) Description of First Embodiment”, and the description also applies to the present embodiment. Specifically, the data acquisition device 1 includes the imaging element 100 including the signal acquisition unit 110 that acquires image signals by imaging a biological sample at two or more different time points, the information processing unit 101 that extracts a feature amount from the image signal and generates data related to the biological sample on the basis of the feature amount, and the output control unit 150 that causes the data related to the biological sample to be output to the outside of the imaging element. The signal acquisition unit 110, the information processing unit 101, and the output control unit 150 may be arranged in a single chip.

Furthermore, the data acquisition device 1 may include hardware necessary for a computer, such as a central processing unit (CPU), a read only memory (ROM), a random access memory (RAM), and a hard disk drive (HDD). The CPU loads the program of the present technology stored in the ROM or the HDD into the RAM and executes the program, so that the operation of the data acquisition method as described later can be controlled.

The program may be installed in the data acquisition device 1 via, for example, various storage media (internal memories). Alternatively, the program may be installed via the Internet or the like.

In the present embodiment, the data acquisition device 1 provided with the imaging element 100 may be connected to, for example, an information processing device (for example, a personal computer (PC) or the like) 1050.

The output unit 1060 configured to be capable of outputting data (image data, alert data, and the like) related to the biological sample. The output unit 1060 may include, for example, a display device (display) using liquid crystal, organic electro-luminescence (EL), or the like. The display device can output the data related to the biological sample as image (still image or moving image) data, character data, sound data, or the like. Furthermore, the output unit 1060 may include, for example, a printing device. The printing device may print and output the data related to the biological sample on a print medium such as paper.

The input unit 1070 is, for example, a device that receives an operation by a user. The input unit 1070 may include, for example, a mouse, a keyboard, or a display (in this case, the user operation may be a touch operation on the display). The input unit 1070 can transmit an operation by the user to the data processing device 1 as an electrical signal. The information processing unit 101 of the data processing device 1 can perform various types of processing according to the electrical signal.

### (4) Example of Processing of Data by Imaging Element in First Embodiment

Hereinafter, an example of processing of data by the imaging element 100 according to the present technology will be described in detail, but it is not particularly limited thereto.

### (4-1) First Example of Processing of Data by Imaging Element

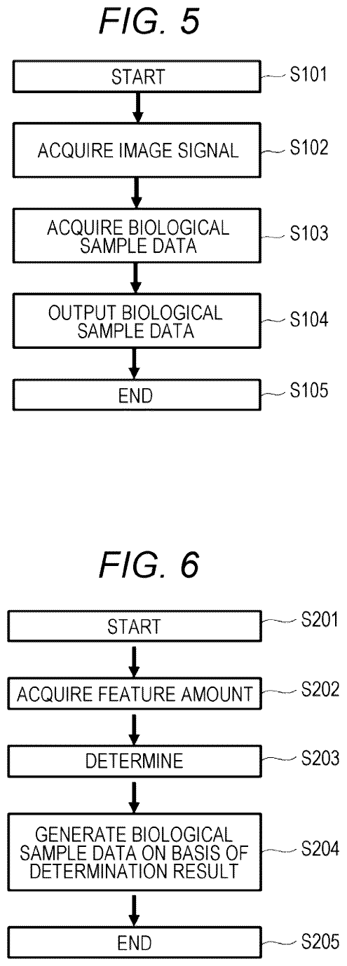

An example of processing of data related to a biological sample by the imaging element 100 included in the data acquisition device 1 will be described below with reference to FIGS. 5 and 6. FIGS. 5 and 6 are an example of an outline of a flowchart for processing data related to a biological sample by the imaging element 100.

The imaging element 100 is as described above with reference to FIG. 1, and includes the signal acquisition unit 110 that can image a biological sample and acquire image signals of the biological sample at two or more different time points, the imaging processing unit 120 that controls imaging processing related to imaging in the signal acquisition unit 110, the information processing unit 101 that extracts a feature amount from the image signals and generates the data related to the biological sample on the basis of the feature amount, and the output control unit 150 that causes the data related to the biological sample to be output to the outside of the imaging element.

### <Embodiment Related to First Example>

In step S101, the imaging element 100 starts acquisition processing of the data related to the biological sample. The imaging element 100 starts to image the biological sample and acquire image signals continuously or over time. The start may be automatic, or may be started by, for example, the user clicking a predetermined processing start button displayed on the display of the output unit.

Note that a learned model may be generated prior to the start of processing of the biological sample data, and the learned model may be stored in a storage unit included in the imaging element 100.

In step S102, the imaging element 100 images the biological sample and acquires an image signal. The imaging element 100 can acquire image signals of the biological sample at two or more different time points. For example, the imaging element 100 may control the imaging processing unit 120, so that the imaging by the signal acquisition unit 110 can be controlled. The imaging element 100 acquires, for example, moving image data or time-lapse image data.

An analog image signal acquired by the signal acquisition unit 110 is converted into a digital image signal by, for example, the imaging processing unit 120, and the digital image signal is transmitted to the information processing unit 101. The information processing unit 101 uses the image signal for generating the data related to the biological sample in step S103 described later.

In step S103, the information processing unit 101 extracts a feature amount from the image signals of the biological sample at two or more different time points, and generates the data related to the biological sample on the basis of the feature amount.

The biological sample is preferably one kind or two or more kinds selected from a cell culture, a fertilized egg, a sperm, nucleic acids, and a biological tissue piece.

The feature amount is preferably any of a feature amount related to a cell culture, a feature amount related to a fertilized egg, a feature amount related to a sperm, a feature amount related to nucleic acids, or a feature amount related to a biological tissue piece.

The data related to the biological sample preferably includes one kind or two or more kinds selected from image data, alert data, flag data, nucleic acid sequence data, and attention amount data. In a case where the data related to the biological sample is image data, the image data may be generated by, for example, the image generation unit 105. Note that, in the present description, data other than image data (for example, alert data, flag data, nucleic acid sequence data, attention data, and the like) among the data related to the biological sample is also referred to as signal data.

In step S104, the output control unit 150 causes the generated data related to the biological sample to be output to the outside of the imaging element. In a case where the data related to the biological sample is not generated, the information processing unit 101 does not have to output data to the outside of the imaging element.

Furthermore, the output data related to the biological sample may be stored in a storage unit or a server inside or outside the data acquisition device. Moreover, it is preferable to store the signal data and the image data associated with the signal data by associating the signal data (for example, alert data, flag data, and the like) with the image data among the data related to the biological sample. Even in a case where any one of the image data or the signal data is output from the output control unit 150, the other associated data can be called and displayed. At this time, it is preferable to output the signal data from the viewpoint that the amount of data to be output can be further reduced.

When the data related to the biological sample is output in step S104, the data acquisition processing can be ended (step S105). Note that the processing of steps S102 to S104 may be repeated again after the data related to the biological sample is output in step S104.

Hereinafter, details of step S103 will be described with reference to FIG. 6.

In step S201 illustrated in FIG. 6, the information processing unit 101 starts the processing of generating the data related to the biological sample in response to, for example, reception of the image signals at two or more different time points acquired in S102.

In step S202, the information processing unit 101 acquires a feature amount from the image signals at two or more different time points of the biological sample. The feature amount may be acquired by the feature amount extraction unit 102. For example, the feature amount extraction unit 102 included in the recognition processing unit 104 extracts changes (differences) of image signals at two or more different time points.

In step S203, the information processing unit 101 determines the state of the biological sample on the basis of the feature amount. The determination of the biological sample can be performed by the state determination unit 103. For example, the state determination unit 103 included in the recognition processing unit 104 can determine that a predetermined event occurs. For example, the recognition processing unit 104 can determine a change (difference) in the image signal at two or more different time points and determine that a predetermined event occurs. Thus, a determination result of the state of the biological sample can be obtained. Furthermore, it is also possible to obtain a determination result of a state before a predetermined event occurs.

In step S204, the data related to the biological sample to be output is generated on the basis of a determination result by determination of the state of the biological sample. The data related to the biological sample preferably includes one kind or two or more kinds selected from image data, alert data, flag data, nucleic acid sequence data, and attention data. The generation of the data related to the biological sample may be performed by the image generation unit 105. Further, the data related to the biological sample other than the image data (for example, signal data such as alert data, and the like) may be generated by the recognition processing unit 104. Furthermore, the information processing unit 101 may generate data related to the biological sample in which image data and other data (for example, signal data) are associated.

The data related to the biological sample is acquired as described above. When the data is acquired, the information processing unit 101 ends the processing in step S103 (step S205).

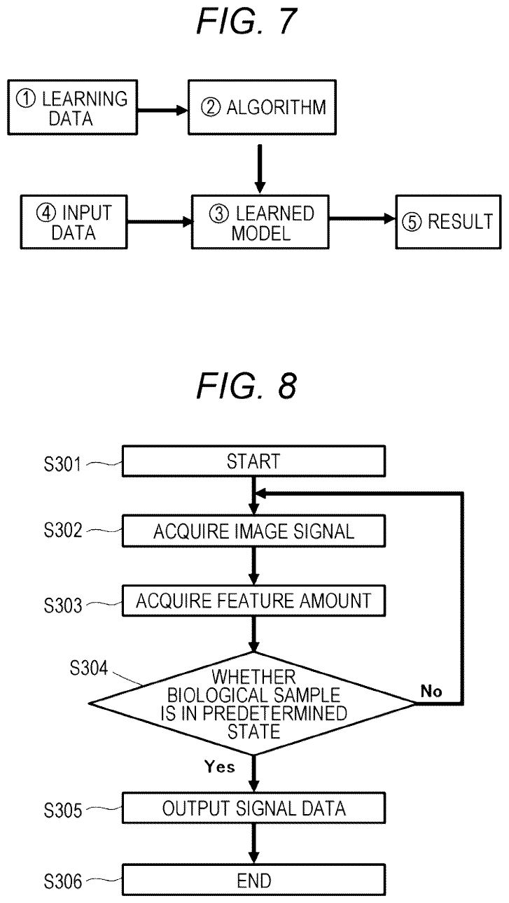

The information processing unit 101 preferably uses a learned model when generating the data related to the biological sample. FIG. 7 is a block diagram schematically illustrating a processing procedure example of the specialized AI that can be used as a learned model in the present technology. The processing using the learned model in the present technology may be performed according to a general processing procedure of a specialized artificial intelligence (AI). The specialized AI uses a learned model generated by causing a predetermined algorithm to perform machine learning on learning data (teacher data). A result is obtained by applying arbitrary input data to the learned model.

In advance, the information processing unit 101 performs conversion or processing on the image data related to the biological sample on the basis of the feature amount related to the biological sample, and sets secondary processed data generated for facilitating analysis by the learning method as teacher data (learning data). The feature amount related to the biological sample at this time may be arbitrarily set by the user, or may be set by a feature amount related to the biological sample derived empirically. Furthermore, the image data related to the biological sample may be obtained by imaging by the signal acquisition unit 110, or may be obtained from the inside of the device or the outside of the device, such as a storage unit or on a server.

Next, the information processing unit 101 can construct the learned model by causing a preset algorithm to perform machine learning using the teacher data. Thus, the information processing unit 101 has a configuration having a learned model.

The algorithm functions as, for example, a machine learning algorithm. The information processing unit 101 may select a single learned model from among learned models constructed from the respective feature amounts, or may select a learned model obtained by combining a plurality of learned models. Furthermore, the learned model is not particularly limited, and the user may select one or more learned models.

The type of the machine learning algorithm is not particularly limited, and may be, for example, an algorithm using a neural network such as a recurrent neural network (RNN), a convolutional neural network (CNN), or a multilayer perceptron (MLP), or an arbitrary algorithm.

Next, the information processing unit 101 can generate the data related to the biological sample to be output from the output control unit 150 by inputting the image signal acquired by the signal acquisition unit 110 to the constructed learned model. Note that the acquired image signal corresponds to the input data of FIG. 7, and the data related to the biological sample to be output corresponds to the result of FIG. 7.

The learned model may be, for example, a learned model generated by deep learning. For example, the learned model may be a multilayer neural network, for example, may be a deep neural network (DNN), and more specifically, may be a convolutional neural network (CNN).

A multilayer neural network may be used as a learned model used for the feature amount extraction unit to extract the feature amount. The multilayer neural network may include an input layer that inputs image data, an output layer that outputs the feature amount of the image data, and at least one intermediate layer provided between the input layer and the output layer.

The multilayer neural network may be used as a learned model used for the state determination unit to generate data related to the biological sample. The multilayer neural network may include an input layer that inputs the feature amount, an output layer that outputs data related to the biological sample based on the feature amount, and at least one intermediate layer provided between the input layer and the output layer.

The image data related to the biological sample that can be acquired by the information processing unit 101 may be image data obtained by imaging by the signal acquisition unit 110, or may be image data of the inside (for example, the storage unit) or the outside (for example, on the network), and is not particularly limited thereto.

Note that a first example according to the present technology can be applied by appropriately employing methods as necessary from the data acquisition methods described in a second example to a seventh example and appropriately combining the methods.

### (4-2) Second Example of Processing of Data by Imaging Element

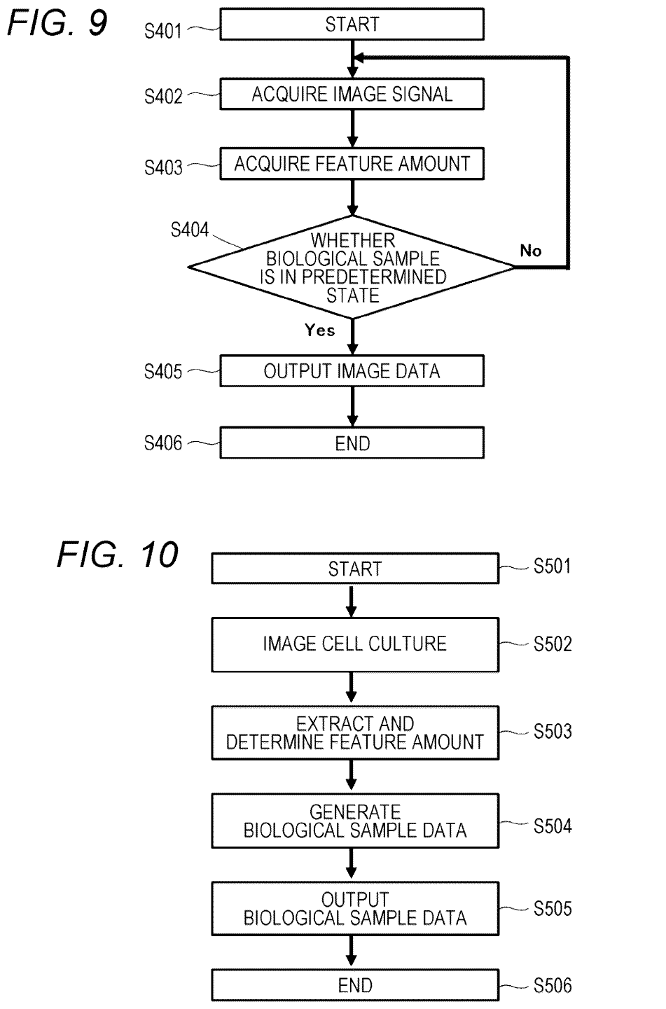

An example of processing of data related to a biological sample by the imaging element 100 included in the data acquisition device 1 will be described below with reference to FIGS. 8 and 9. FIGS. 8 and 9 are an example of an outline of a flowchart for processing data related to the biological sample by the imaging element 100.

The imaging element 100, the signal acquisition unit 110, the imaging processing unit 120, the information processing unit 101, the output control unit 150, and the like related to the second example can employ the above-described (4-1) First Example of Processing of Data by the Imaging Element. An embodiment related to the second example will be described with reference to FIGS. 8 and 9. This makes it possible to reduce the amount of data to be output when the biological sample is imaged.

Note that the second example according to the present technology can be applied by appropriately employing methods as necessary from the data acquisition methods described in the first example and a third example to a seventh example, and appropriately combining the methods.

An embodiment related to a second example A will be described with reference to FIG. 8.

In step S301, the imaging element 100 starts processing the data related to the biological sample. The imaging element 100 starts to obtain image signals continuously or over time by imaging the biological sample.

In step S302, the information processing unit 101 acquires image signals of the biological sample at two or more different time points from the signal acquisition unit 110 via the imaging processing unit 120.

In step S303, the information processing unit 101 extracts the feature amount from the image signals of the biological sample at two or more different time points.

In step S304, the information processing unit 101 determines whether or not the state of the biological sample has reached a predetermined state. The information processing unit 101 may be a determination of whether or not the predetermined state can be reached. The predetermined state may also include an elapsed time and the like. The information processing unit 101 may generate the data related to the biological sample from the feature amount or the predetermined state using the learned model.

In a case where the information processing unit 101 determines that the predetermined state has not been reached, the processing returns to step S302 to acquire image signals.

In a case where the information processing unit 101 determines that the predetermined state has been reached, the processing proceeds to step S305, and signal data (for example, alert data or the like) related to the biological sample is generated on the basis of the feature amount. In step 305, the information processing unit 101 outputs the signal data related to the biological sample to the outside of the imaging element.

When the data related to the biological sample is output in step S304, the data acquisition processing can be ended (step S305). Note that the processing of steps S302 to S304 may be repeated again after the data related to the biological sample is output in step S304.

An embodiment related to a second example B will be described with reference to FIG. 9.

In step S401, the imaging element 100 starts processing the data related to the biological sample. The imaging element 100 starts to continuously or temporally obtain image signals acquired by imaging the biological sample.

In step S402, the information processing unit 101 obtains image signals of the biological sample at two or more different time points from the imaging processing unit 120.

In step S403, the information processing unit 101 extracts the feature amount from the image signals of the biological sample at two or more different time points.

In step S404, the information processing unit 101 determines whether or not the state of the biological sample has reached a predetermined state. The information processing unit 101 may determine whether or not the predetermined state can be reached. The information processing unit 101 may generate the data related to the biological sample using the learned model.

In a case where the information processing unit 101 determines that the predetermined state has not been reached, the processing returns to step S402 to acquire image signals.

In a case where the information processing unit 101 determines that the predetermined state has been reached, the processing proceeds to step S405, and image data related to the biological sample is generated on the basis of the feature amount. Furthermore, in a case where the information processing unit 101 determines that the predetermined state has been reached, the compression rate of the image data may be changed according to the degree of importance of the image data. For example, in a case where the importance of the image data is high, the image data may not be compressed or the compression rate of the image data may be lowered, or in a case where the importance of the image data is low, the compression rate of the image data may be raised. In addition, image data of a region other than the necessary region may be compressed. Further, when the image data is generated, time data such as an elapsed time, coordinate position data such as a place, and image data associated with this data may be generated. Furthermore, at this time, signal data (for example, alert data or the like) can also be generated.

In step 405, the information processing unit 101 outputs data including image data related to the biological sample to the outside of the imaging element. The data related to the biological sample may include signal data.

When the data related to the biological sample is output in step 404, the data acquisition processing may be ended (step S405). Note that the processing of steps S402 to S404 may be repeated again after the data related to the biological sample is output in step S404.

### (4-3) Third Example of Processing of Data Related to Cell Culture by Imaging Element

Hereinafter, as a third example of the present technology, processing of data related to a cell culture by the imaging element 100 will be described with reference to FIG. 10.

The information processing unit 101 according to the present technology acquires image signals by imaging a cell culture at two or more different time points, extracts the feature amount from the acquired image signals, and generates data related to the cell culture on the basis of the feature amount.

The information processing unit 101 can determine the state of the biological sample on the basis of the feature amount of the cell culture.

In a case where it is determined that the cell culture is in the predetermined state, the information processing unit 101 can generate and output data related to the cell culture. In a case where it is determined that the predetermined state has been reached or can be reached, the information processing unit 101 may perform work processing related to the cell culture on the basis of the determination result when it is possible to be output, or the user may determine the output data related to the cell culture and input the work processing related to the cell culture. As the work processing related to the cell culture, for example, one kind or two or more kinds can be selected from end of culture, subculture, drug addition, cell sorting, cell recovery, and the like, or a combination thereof (for example, drug addition followed by cell sorting/recovery).

Furthermore, the information processing unit 101 can take, for example, as the data related to the biological sample: modeling of form of a culture vessel (for example, a petri dish, a bottle, a chamber, and the like), presence or absence of a culture vessel, and the like; presence or absence of cell culture, the number of culture vessels, and culture period per culture vessel; modeling of form of a cell, dotting, or the like; and the number, proliferation, disappearance, form, tracking, and motion of cells.

Conventionally, when monitoring is performed in real time with an imager such as a CCD or a CMOS, there is a problem that image data becomes enormous.

On the other hand, by using the imaging element 100 according to the present technology, the amount of image data can be reduced. Since the present technology can reduce the amount of data, increasing the number of monitoring subjects or the like in real time can be performed for a long period of time. Furthermore, by using the imaging element 100 according to the present technology, it is possible to automate cell culture, such as cell sorting, passage, and drug administration timing.

The cell culture in the present technology may include a tissue, a cell, a virus, a bacterium, a culture solution, a metabolite, and the like. Tissues include two-dimensionally or three-dimensionally cultured tissues, spheroids, and cell masses. Moreover, cells include stem cells, induced pluripotent stem cells (iPS) cells, cancer cell lines, genetically engineered cells, and the like.

The feature amount related to cells is not particularly limited, and examples thereof include shape, number of cells, density, proliferation speed, activity, movement, and the like, and one kind or two or more kinds thereof can be selected therefrom.

Furthermore, the feature amount related to the culture solution is not particularly limited, and examples thereof include the number of foreign substances in the culture medium, nutrient components (for example, proteins, carbohydrates, lipids, minerals, and the like) of the culture medium, the content of each nutrient component, carbon dioxide concentration, oxygen concentration, temperature, atmospheric pressure, gas atmosphere, light transmission, light scattering, light absorption, pH, pH responders, and the like, and one kind or two or more kinds thereof can be selected. The foreign substance is not particularly limited, and examples thereof include microorganisms (for example, fungi (for example, bacteria, fungi, or the like), viruses, mycoplasmas, and the like).

The extraction of the feature amount related to the cell culture is not particularly limited, but can be performed, for example, on the basis of a change (difference) in image signals related to the cell culture imaged at two or more different time points. More specifically, in a case where a change (difference) in the image signals at two or more different time points occurs, the change (difference) can be extracted as the feature amount related to the cell culture. Thus, it is possible to acquire the feature amount related to the cell culture.

A predetermined state related to the biological sample can be determined on the basis of the feature amount related to the cell culture. The predetermined state is not particularly limited, and examples thereof include a state in which a predetermined cell density is reached or can be reached, a state in which a foreign substance is generated or can be generated, and the like.

Then, in a case where it is determined that a state where the predetermined cell density has been reached or a state where the predetermined cell density can be reached has been reached, data related to the cell culture is generated and output. The data related to the cell culture is not particularly limited, and examples thereof include an alert of cell density, an alert of passage time, an alert of drug administration, image data related to the generated cell culture, and the like, and can include one kind or two or more kinds selected therefrom.

Furthermore, in a case where it is determined that a foreign substance has occurred or has reached a state in which the foreign substance can occur, data related to the cell culture is generated and output. The data related to the cell culture include is not particularly limited, and examples thereof include an alert of foreign substance generation, an alert of passage time, an alert of drug administration, a culture solution exchange alert, image data related to the generated cell culture, and the like, and can include one kind or two or more kinds selected therefrom.

According to the present technology, the cell culture can be managed. Furthermore, in the present technology, the feature amount related to the cell culture may be determined using a learning model for a plurality of image signals acquired by imaging the cell culture.

According to the present technology, it is possible to determine the feature amount related to the cell culture from image signals at two or more different time points of the cell culture on imaging pixels, and generate data related to the cell culture. Thus, it is not necessary to continuously output a large amount of image data to the outside of the imaging element, and the amount of data transfer to the outside of the imaging element can be reduced.

Furthermore, the feature amount related to the cell culture is not particularly limited, and may include, for example: feature amounts related to cells, such as the number of cells, cell division (for example, the number, speed, shape, and the like), cell activity degree (for example, enzymes, metabolites, and the like), and movement of cells; feature amounts related to culture solution such as culture solution composition and the number of microorganisms; and the like, and one kind or two or more kinds thereof may be selected. When the feature amount is detected, coloration method detection, fluorescence method detection, antigen-antibody reaction detection, and the like, or a combination thereof may be appropriately used as necessary.

According to the present technology, in a case where culture management such as contamination countermeasures is performed, for example, it is possible to recognize a cell having a characteristic different from that of a cell originally desired to be cultured as a foreign substance, and present an alert. Furthermore, at this time, a difference between the original cell culture and the foreign substance may be determined using a learning model. Examples of the foreign substance include, but are not limited to, microorganisms such as bacteria, fungi, microplasma, viruses, and the like.

In the culture management, examples of the difference between the cell and the foreign substance include a size, a shape, a growth rate, a division rate, a movement, an activity, an existence place, light scattering, an internal structure, and the like, and one kind or two or more kinds selected therefrom can be used as the feature amount of the cell culture, but the difference is not particularly limited thereto.

Furthermore, according to the present technology, before or when an event occurs, the information processing unit 101 may acquire image signals at two or more different time points, generate data related to the cell culture on the basis of the image signals, and output the data as data related to the cell culture (for example, image data such as cultured cells, alert data, and the like) to the outside of the imaging element.

Before the event occurs is not particularly limited, and examples thereof include before drug addition, before passage, before cell sorting, before cell addition, before end of cell culture, and the like.

When an event occurs is not particularly limited, and examples thereof include a time when a set time is reached; a time when the cell culture reaches a desired cell density or cell number; a time when a foreign substance is generated in culture solution; and a time when a drug is administered, and the like.

According to the present technology, the amount of data to be output can be further reduced.

A third example A of processing of data related to the cell culture by the imaging element will be exemplified below, but it is not particularly limited thereto (see FIG. 10).

In step S501, culturing of the cell culture is started, and monitoring of the cultured cell is started.

In step S502, the information processing unit 101 controls the signal acquisition unit 110 to acquire image signals of cell cultures at two or more different time points.

In step S503, the information processing unit 101 extracts a feature amount of the cell culture from the image signals at two or more different time points of the cell culture, and generates data related to the cell culture on the basis of the feature amount. At this time, a learned model may be used.

In step S504, the output control unit 150 outputs the data related to the cell culture to the outside of the imaging element.

When the data related to the cell culture is output in step S504, the data acquisition processing can be ended (step S505). Note that, in step S504, after the data related to the cell culture is output, the processing of steps S502 to S504 may be repeated again.

In step S505, work processing on the cell culture is performed on the basis of the data related to the cell culture. The user may input or instruct work processing related to the cell culture on the basis of the output data related to the cell culture. Furthermore, a work processing unit related to the cell culture may be provided, various work processing methods may be set in advance in the work processing unit, the data related to the cell culture may be transmitted to the work processing unit related to the cell culture, and various work processing of the cell culture may be performed in the work processing unit related to the cell culture on the basis of the transmitted data, thereby enabling automatic work processing.

A third example B of processing of data related to the cell culture by the imaging element will be exemplified below, but the processing is not particularly limited thereto (see FIG. 10).

In step S501, culturing of the cell culture is started, and monitoring of the cultured cell is started.

In step S502, the information processing unit 101 controls the signal acquisition unit 110 to acquire image signals of cell cultures at two or more different time points.

In step S503, the information processing unit 101 extracts feature amounts of the cell number and the cell density from the image signals, and generates data related to the cell number and the cell density on the basis of the feature amounts. At this time, a learned model may be used.

In step S504, the information processing unit 101 determines the state of the cell culture on the basis of data related to the number of cells and the cell density. The state of the cell culture at this time is preferably a state where a predetermined number of cells and/or a predetermined cell density can be reached or has been reached. Data related to the cell culture including a state where it can be reached or has been reached is generated and output to the outside of the imaging element. In step S504, as the data, alert data may be continuously output to the outside of the imaging element. Further, image data obtained by imaging immediately before the reaching or at the time of the reaching may be output to the outside of the imaging element. Furthermore, the alert data and data including the image data may be output to the outside of the imaging element.

When the data related to the cell culture is output in step S504, the data acquisition processing can be ended (step S505). Note that, in step S504, after the data related to the cell culture is output, the processing of steps S502 to S504 may be repeated again.

According to the present technology, the amount of data to be output can be further reduced. Furthermore, since the image data and the alert data necessary for the work processing are appropriately output, the user can easily perform the work processing related to the cell culture.

For example, in a case of determining continuation of the culture, the user may determine the end of culture or the passage of the cells on the basis of the output data related to the cell culture, and perform input of an end or the like.

For example, in a case of determining drug addition to the cell culture, the user can perform the drug addition to the cell culture on the basis of the output data related to the cell culture.

For example, in a case of determining cell sorting or cell recovery, the user can perform the cell sorting or cell recovery on the cell culture on the basis of the output data related to the cell culture.

In a case of determining drug addition to the cell culture and then cell sorting/recovery, the user can perform the drug addition to the cell culture on the basis of the output data related to the cell culture. Moreover, the cell culture may be continued, and steps similar to steps S501 to S504 in the third example B described above may be performed to perform the cell sorting/recovery.

Note that a work processing unit related to the cell culture may be provided, various work processing methods may be set in advance in the work processing unit, and the work processing unit may perform work processing similar to work processing performed by the user instead of the user. For example, the data related to the cell culture is transmitted to the work processing unit related to the cell culture, and the work processing unit related to the cell culture can automatically perform various work processes of the cell culture on the basis of the transmitted data.

Furthermore, the third example according to the present technology can be applied by appropriately employing methods as necessary from the data acquisition methods described in the first example and the second example and a fourth example to a seventh example, and appropriately combining the methods.

### (4-4) Fourth Example of Processing of Data Related to Fertilized Egg by Imaging Element

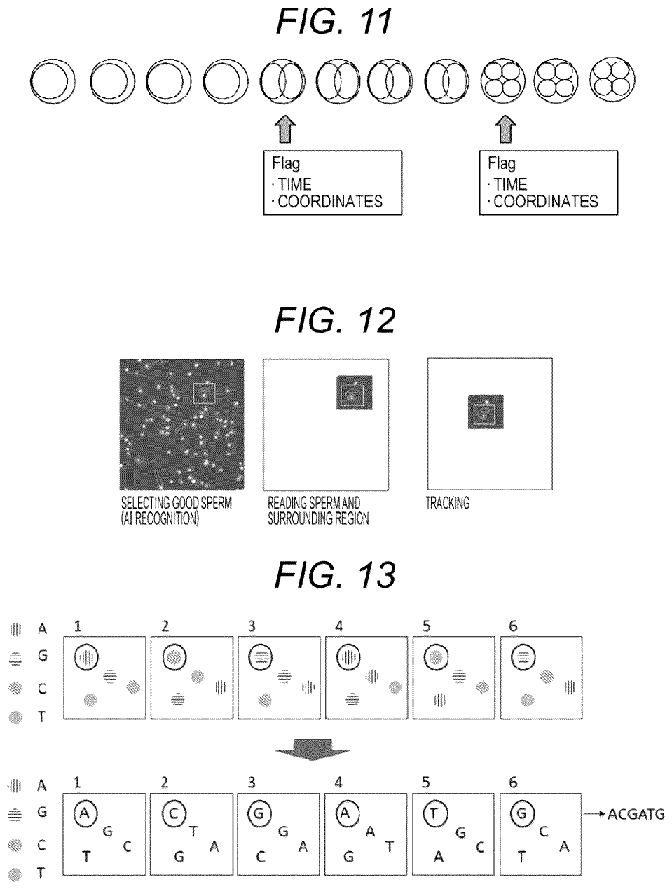

Hereinafter, as a fourth example of the present technology, processing of data related to a fertilized egg by the imaging element will be described with reference to FIG. 11.

The information processing unit 101 according to the present technology acquires image signals by imaging a fertilized egg at two or more different time points, extracts the feature amount from the acquired image signals, and generates data related to the fertilized egg on the basis of the feature amount.

The information processing unit 101 can determine the state of the biological sample on the basis of the feature amount of the fertilized egg.

In a case where it is determined that the fertilized egg has reached a predetermined state, the information processing unit 101 can generate and output data related to the fertilized egg. In a case where it is determined that the predetermined state has been reached, the information processing unit 101 may perform work processing on the fertilized egg on the basis of a determination result when it is possible to be output, or the user may determine the output data related to the fertilized egg and input the work processing on the fertilized egg. As the work processing related to the fertilized egg, for example, one kind or two or more kinds can be selected from cell division, end of culture, subculture, drug addition, cell sorting, cell recovery, and the like, or a combination thereof (for example, cell division followed by cell sorting/recovery).

The information processing unit 101 can tag the image data according to the change amount (difference) of the image signals acquired at two or more different time points, and output the tagged image data to the outside of the imaging element as data related to the fertilized egg. Image data obtained by imaging before this may be held in the memory. For example, it is possible that: two continuously imaged images are stored in the memory as image data; two continuously imaged images are compared, and in a case where a predetermined state is exceeded (or not exceeded), tag data is added to the image data; tag-attached image data is output to the outside of the imaging pixel as data related to the fertilized eggs; or untagged image data (for example, image data having no change (difference)) is not output to the outside of the imaging element or is generated as data (for example, alert data or the like) having a small amount other than the image data, and is output to the outside of the imaging element.

Note that, for example, tagging can be performed on, but is not limited to, image data at a time point at which the state of the cell culture has changed or before the state of the cell culture can change, such as a time point at which a cell such as a fertilized egg is divided; and coordinates (field of view with the speed of stage movement or operation of the endoscope being changed) receiving attention by a pathologist or a surgeon; extraction of image change point during line scan, and the like.

Conventionally, when monitoring a fertilized egg, image data obtained by monitoring is continuously output to an external server and image data stored in the server is analyzed, but since the amount of image data is large and the image data is continuously output to the outside, the output image data and the amount of image data to be processed become large.

On the other hand, the amount of data can be reduced by extracting the feature amount (feature point, time, and the like) using the imaging element 100 according to the present technology. The amount of data may be reduced simultaneously with storage of the image data. Since the present technology can reduce the amount of data, increasing the number of monitoring subjects or the like in real time can be performed for a long period of time. Furthermore, by using the imaging element 100 according to the present technology, it is possible to automate cell culture such as division of a fertilized egg, cell sorting, passage, and timing of drug administration.

The biological sample including a fertilized egg in the present technology may include a fertilized egg, a culture solution, and the like. The feature amount related to the fertilized egg is not particularly limited, and examples thereof include division (for example, a division shape, a division speed, and the like), a shape of the fertilized egg, a degree of activity, and the like, and one kind or two or more kinds can be selected from these. The culture solution is similar to the culture solution in the cell culture described above.

The extraction of the feature amount related to the fertilized egg is not particularly limited, but can be performed, for example, on the basis of a change (difference) in image signals related to the fertilized egg acquired at two or more different time points (see, for example, FIG. 11). More specifically, in a case where a change (difference) in the image signals at two or more different time points occurs, the change (difference) can be extracted as the feature amount related to the fertilized egg. Thus, the feature amount related to the fertilized egg can be acquired.

A predetermined state related to the biological sample can be determined on the basis of the feature amount related to the fertilized egg. The predetermined state is not particularly limited, and examples thereof include a state in which a predetermined division process is reached or can be reached, a state in which a foreign substance is generated or can be generated, and the like.

Then, in a case where it is determined that a state where the predetermined division process has been reached or a state where the predetermined division process can be reached has been reached, the information processing unit 101 generates and outputs data related to the fertilized egg. The data related to the fertilized egg is not particularly limited, and examples thereof include an alert of division process, an alert of cell sorting/recovery, an alert of culture solution exchange, an alert of drug administration, and image data related to the generated fertilized egg, and the like, and can include one kind or two or more kinds selected therefrom.

According to the present technology, a fertilized egg division process can be managed. Furthermore, in the present technology, a feature amount related to the fertilized egg may be determined using a learning model for a plurality of image signals acquired by imaging a division process of the fertilized egg.

According to the present technology, it is possible to determine the feature amount related to the fertilized egg from image signals at two or more different time points of the fertilized egg on the imaging element, and generate data related to the fertilized egg on the basis of a determination result. Thus, it is not necessary to continuously output a large amount of image data to the outside of the imaging element, and the amount of data transfer to the outside of the imaging element can be reduced.

In addition, the feature amount related to the fertilized egg is not particularly limited, and may include, for example: feature amounts related to the fertilized egg, such as division of the fertilized egg (for example, the number of divisions, division speed, division shape, and the like), and cell activity degree (for example, enzymes, metabolites, and the like); feature amounts related to culture solution such as culture solution composition and the number of microorganisms; and the like, and one kind or two or more kinds thereof may be selected.

Furthermore, according to the present technology, in a case where fertilized egg management such as a division process is performed, the information processing unit 101 may acquire image signals at two or more different time points of the fertilized egg, generate data related to the fertilized egg on the basis of the image signals, and output the data related to the fertilized egg (for example, tagged image data, alert data, and the like) to the outside of the imaging element.

According to the present technology, in a case where the fertilized egg management such as a division process is performed, for example, division time points can be determined on the basis of image signals acquired at two or more different time points, and data related to the fertilized egg can be generated on the basis of the determination result.

The data related to the fertilized egg may include image data obtained by imaging at the time of division in addition to the alert data. The information processing unit 101 may generate image data associated with data of a flag at the time of division. The flag may include an elapsed time in the division process of the fertilized egg, coordinates of the fertilized egg, and the like. The information processing unit 101 can change the compression rate between the image data with the flag and the image data without the flag, and can reduce the amount of data to be output to the outside of the imaging element by increasing the compression rate of the image data without the flag. Moreover, the image data with the flag may be stored therein.

Furthermore, the image data with the flag may be output to the outside of the imaging element, and the image data without the flag may be prohibited to be output to the outside of the imaging element or may be generated as data (for example, alert data or the like) having a small amount other than the image data, and then output to the outside of the imaging element.

According to the present technology, it is not necessary to store, read, and analyze a large amount of image data, and it is also possible to analyze a division time point inside the device and appropriately store the division time point. Moreover, it is possible to reduce storage and analysis calculation amount outside a server or the like.

According to the present technology, the amount of data to be output can be further reduced. Furthermore, since the image data and the alert data necessary for the work processing are appropriately output, the user can easily perform the work processing related to the fertilized egg.

Note that the fourth example according to the present technology can be applied by appropriately employing methods as necessary from the data acquisition methods described in the first example to the third example and a fifth example to a seventh example, and appropriately combining the methods.

### (4-5) Fifth Example of Processing of Data Related to Sperm by Imaging Element

Hereinafter, as a fifth example of the present technology, processing of data related to sperm by the imaging element will be described with reference to FIG. 12.

The information processing unit 101 according to the present technology acquires image signals by imaging a biological sample containing sperm at two or more different time points, extracts the feature amount from the acquired image signals, and generates data related to the sperm on the basis of the feature amount.

The information processing unit 101 can determine the state of the biological sample on the basis of the feature amount of the sperm.

In a case where it is determined that the sperm is in the predetermined state, the information processing unit 101 can generate and output data related to the sperm. In a case where it is determined that the state has reached the predetermined state, the information processing unit 101 may perform work processing on the sperm on the basis of a determination result when it is possible to be output, or the user may determine the output data related to the sperm and input the work processing on the sperm. As the work processing related to sperm, for example, one kind or two or more kinds can be selected from sperm cell sorting, sperm cell recovery, drug addition, and the like.

Conventionally, when monitoring is performed in real time with an imager such as a CCD or a CMOS, there is a problem that image data becomes enormous.

On the other hand, by using the imaging element 100 according to the present technology, the amount of image data can be reduced. Since the present technology can reduce the amount of data, increasing the number of monitoring subjects or the like in real time can be performed for a long period of time.

The biological sample including sperm in the present technology may include sperm, a culture solution, or the like. The feature amount related to sperm is not particularly limited, and examples thereof include movement of sperm, sperm shape, activity, and the like, and one kind or two or more kinds thereof can be selected. The culture solution is similar to the culture solution in the cell culture described above.

The extraction of the feature amount related to the sperm is not particularly limited, but can be performed, for example, on the basis of a change (difference) in image signals related to the sperm acquired at two or more different time points (see, for example, FIG. 12). More specifically, in a case where a change (difference) in the image signals at two or more different time points occurs, the change (difference) can be extracted as the feature amount related to the sperm. Thus, the feature amount related to the sperm can be acquired.

It is possible to determine whether or not a predetermined state related to the biological sample has been reached on the basis of the feature amount related to the sperm. The predetermined state is not particularly limited, and examples thereof include a favorable state of sperm, a state in which a foreign substance is generated or can be generated, and the like.

In a case where it is determined that the sperm has reached a favorable state, data related to the sperm is generated and output. The data related to the sperm is not particularly limited, and examples thereof include an alert of cell sorting/recovery, an alert of drug administration, image data related to the generated sperm, and the like, and can include one kind or two or more kinds selected therefrom.

The present technology enables management of sperm selection. Furthermore, in the present technology, a feature amount related to sperm may be determined using a learning model for a plurality of image signals acquired by imaging a biological sample including sperm.

According to the present technology, the feature amount related to the sperm is determined from the image signals of the biological sample containing the sperm at two or more different time points on the imaging element, and the data related to the sperm is generated, so that it is not necessary to continuously output a large amount of image data to the outside of the imaging element, and the amount of data to the outside of the imaging element can be reduced.

Furthermore, the feature amount related to the sperm is not particularly limited, and may include, for example: feature amounts related to sperm such as sperm activity (for example, sperm count, sperm movement speed, sperm shape, and the like) and cell activity degree (for example, enzymes, metabolites, and the like); feature amounts related to culture solution such as culture solution composition and the number of microorganisms; and the like, and one kind or two or more kinds thereof may be selected.

According to the present technology, in a case where sperm selection is managed, the information processing unit 101 may acquire image signals of a biological sample containing sperm at two or more different time points, generate data related to the sperm on the basis of the image signals, and output the data related to the sperm (for example, tagged image data, alert data, and the like) to the outside of the imaging element. For example, a favorable sperm may be determined for in vitro fertilization and data related to the sperm may be generated.

The data related to the sperm may include image data obtained by imaging the selected sperm in addition to the alert data. The information processing unit 101 may determine a sperm favorable for in vitro fertilization, and generate image data of only a region of the sperm or image data including only the region and peripheral pixels on the basis of a determination result. Furthermore, the information processing unit 101 may track the determined sperm and generate coordinate position data from the coordinate position where the sperm exists. At this time, it is preferable to cut out the image data of only the region of the sperm or the image data including only the region and its peripheral pixels, associate the cut out image data with coordinate position data where the sperm exists, and generate the coordinate position data and the image data associated with the coordinate position. Furthermore, a region other than the image data of only the region of the sperm may be deleted, or a region other than the image data including only the region of the sperm and its peripheral pixels may be deleted. The generated data related to the sperm is output to the outside of the imaging element.

According to the present technology, it is possible to select a favorable sperm inside the device and to obtain the image data of only the region of the sperm or the image data including only the region and its peripheral pixels. Thus, it is not necessary to output a large amount of image data of the entire observation region. Furthermore, according to the present technology, the amount of data to be output can be further reduced. Furthermore, since the image data and the alert data necessary for work processing are appropriately output, the user can easily perform processing of the sperm.

Note that the fifth example according to the present technology can be applied by appropriately employing a method as necessary from the data acquisition methods described in the first to fourth examples, a sixth example, and a seventh example, and appropriately combining the methods.

### (4-6) Sixth Example of Processing of Data Related to Nucleic Acid by Imaging Element

Hereinafter, as a sixth example of the present technology, processing of data related to nucleic acids by the imaging element will be described with reference to FIG. 13.

The information processing unit 101 according to the present technology images a spot of nucleic acids to acquire an image signal, extracts a feature amount from the acquired image signal, and generates data related to the nucleic acids on the basis of the feature amount.

The information processing unit 101 can determine the state of the biological sample on the basis of the feature amount of the nucleic acids. As a step performed before determination, it is preferable to divide a spot emitting a signal in the acquired image signal into a region for each spot. The information processing unit 101 can generate and output nucleic acid sequence data in a case where it is determined that a predetermined state regarding the nucleic acids has been reached.

The feature amount related to the nucleic acids is not particularly limited. Examples thereof include a wavelength of a spot, a fluorescence spectrum, an absorption spectrum, an optical characteristic, a fluorescence wavelength, an area, luminance, a distance from a center, circular extraction (hough conversion), and the like, and one kind or two or more kinds thereof can be selected.

Furthermore, the information processing unit 101 can create image data by excluding image data of a region other than the spot.

The information processing unit 101 can convert the acquired image signal into nucleic acid sequence data of AGCT on the basis of the feature amount related to the nucleic acids such as the fluorescence wavelength, the fluorescence spectrum, and the fluorescence intensity. In the present description, the “feature amount related to nucleic acids” includes both a feature amount related to the nucleic acids themselves and a feature amount related to a substance labeled in the nucleic acids (for example, a fluorescent dye or the like), and may be either one or both of them.

It is preferable that the information processing unit 101 can convert the fluorescence signal intensity and the fluorescence wavelength more than or equal to the set threshold value into the type of nucleic acids on the basis of the acquired image signal. For example, in a case where the number of nucleic acids is calculated from a subject spot, the calculation can be performed on the basis of [area or luminance of subject spot]: [area or luminance of spot in a case of one base (reference (threshold) 1)].

In this manner, the information processing unit 101 can determine, for example, the type and/or number of nucleic acids on the basis of the acquired image signal. The information processing unit 101 can generate the nucleic acid sequence data on the basis of the determined type and/or number of nucleic acids.

Thus, by converting the acquired image signal into data such as characters of AGCT, the amount of data can be compressed, and the amount of data can be reduced. Furthermore, according to the present technology, the amount of data to be output can be further reduced.

In addition, by setting the starting point in the image signal, the coordinate position of a fluorescence signal in the image signal is easily set, and the order of the nucleic acid sequence data of the nucleic acids is easily clarified when or after conversion into the nucleic acid sequence data of the nucleic acids. For example, the image data may not be arranged two-dimensionally, and may be sequentially arranged one-dimensionally by simply assigning spot numbers.

Since conventional nucleic acid sequence analysis methods transfer image data as it is, there is a problem that the amount of data becomes enormous and increases a load of data transfer. Since the amount of data increases in this manner, it is necessary to reduce the imaging frequency, limit the imaging period, and restrict monitoring subject samples.

On the other hand, by using the imaging element 100 according to the present technology, it is possible to convert image data obtained by a conventional nucleic acid sequence analysis method into nucleic acid sequence data, and reduce the load of data transfer, and thus improvement in speed can be expected.

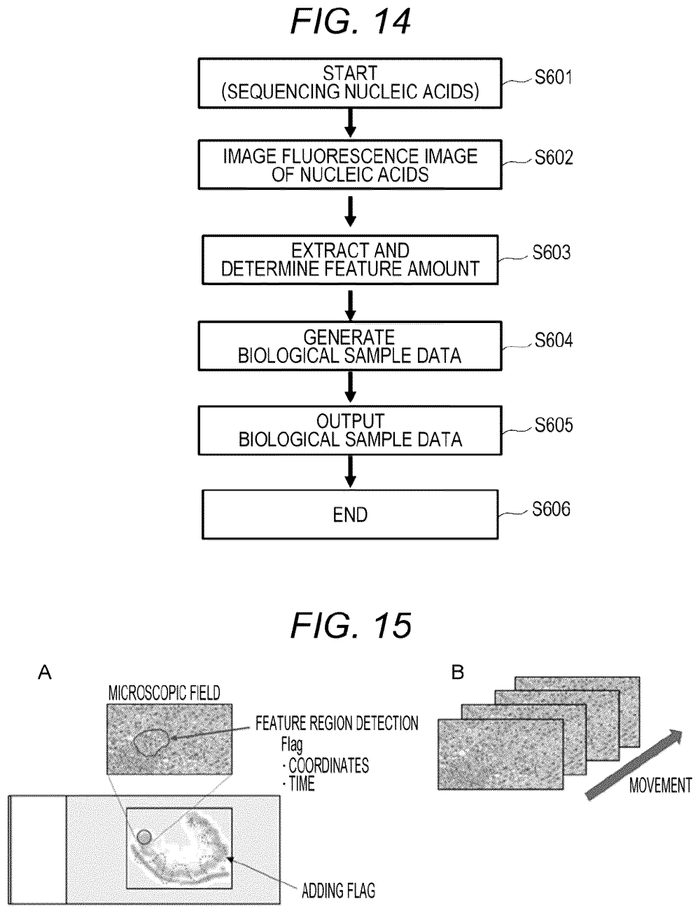

An embodiment related to the fifth example will be described with reference to FIG. 14.

In step S601, the information processing unit 101 starts sequencing of the nucleic acids.

In step S602, the information processing unit 101 performs a fluorescent labeling method and images a fluorescence image including a plurality of fluorescence spots at two or more different time points to acquire image signals related to the fluorescence image.

In step S603, the information processing unit 101 extracts a feature amount (for example, fluorescence wavelength, fluorescence spectrum, fluorescence intensity, fluorescence region, and the like) of the fluorescence spots from the acquired image signal. The information processing unit 101 can extract a signal equal to or more than a threshold value, a spot position, or the like as a feature amount. For example, one of the AGCTs can be set on the basis of a fluorescence spot wavelength. When it is set that a predetermined spot intensity of 1 is one base, in a case where there is an intensity twice this intensity, a double thereof can be set as the number of bases, and for example, in a case where an intensity twice the spot intensity of a nucleic acid A is detected, the fluorescence spot can be determined to have two bases of A, such as AA. In addition, the type, number, and sequence order of bases can be similarly determined by a fluorescence area. Furthermore, the type, number, and sequence order of bases may be determined by combining the fluorescence spot and the fluorescence area.

In step S604, the information processing unit 101 generates data related to the sequence of the nucleic acids on the basis of the feature amount of the fluorescence spot. By dividing each fluorescence spot and setting the starting point, two-dimensional to one-dimensional data can be obtained, and the information processing unit 101 can further set the sequence of the nucleic acids in order.

Furthermore, the information processing unit 101 may create data related to the sequence of the nucleic acids while extending the sequence of the nucleic acids with ACGATG or the like as illustrated in FIG. 13 by repeating S602 to S604 and S603 to S604.

In step S605, the information processing unit 101 outputs data related to the sequence of the nucleic acids to the outside.

When the data related to the sequence of the nucleic acids is output in step S605, the data acquisition processing can be ended (step S606). Note that, in step S605, after the data related to the sequence of the nucleic acids is output, the processing of steps S602 to S604 may be repeated again.

By displaying the data related to the sequence of the nucleic acid to the user, the user can determine whether to continue performing the fluorescent labeling method, and can input this determination. Furthermore, the work processing unit related to the fluorescent labeling method may determine whether to perform this.

According to the present technology, the amount of data to be output can be further reduced. Furthermore, since the image data and the alert data necessary for the work processing are appropriately output, the user can easily perform data processing on the nucleic acids.

Note that the sixth example according to the present technology can be applied by appropriately employing a method as necessary from the data acquisition methods described in the first example to the fifth example and the seventh example, and appropriately combining the methods.

(4-7) Seventh Example of Processing of Data related to Biological Tissue Piece by Imaging Element

Hereinafter, a seventh example of the present technology will be described with reference to FIG. 15.

The information processing unit 101 according to the present technology acquires image signals by imaging a biological sample including a biological tissue piece at two or more different time points, extracts the feature amount from the acquired image signal, and generates data related to the biological tissue piece on the basis of the feature amount. Examples of the feature amount include an attention amount (for example, an attention time, an attention region, the number of times of attention, and the like).

The information processing unit 101 can determine the state of the biological sample including the biological tissue piece on the basis of the feature amount.

It is possible to determine whether or not a predetermined state related to the biological sample is reached on the basis of the feature amount related to the biological tissue piece. Examples of the predetermined state include an attention amount.

In a case where it is determined that the state has reached a predetermined attention amount, the information processing unit 101 generates and outputs data related to the biological tissue piece. The data related to the biological tissue piece is not particularly limited, but may be, for example, attention data. Examples of the attention data include an alert or image data related to an image region having a large attention amount (the number of observations, an observation time, or the like), an alert or image data related to an imaging frame having a large attention amount (slow moving speed, the number of times, an observation time, or the like), and the like, and can include one kind or two or more kinds selected therefrom.

According to the present technology, observation related to the biological tissue piece can be managed. Furthermore, in the present technology, the biological tissue piece may be imaged, and a feature amount related to the biological tissue piece may be determined using a learning model for a plurality of acquired image signals.

According to the present technology, the amount of data to the outside of the imaging element can be reduced.

For example, as a seventh example A, the information processing unit 101 can detect a feature region in an image of microscopic observation and add a flag to a visual field including the feature region when the user performs microscopic observation of a specimen in a wide visual field.

The information processing unit 101 can change the compression rate between the image data of only a feature region with the flag or the image data including only the feature region and peripheral pixels and image data of a region other than the image data. By compressing image data of a region other than the image data, data to be output to the outside can be reduced. Moreover, the information processing unit 101 may have the image data with the flag stored inside, and may output only image data with the flag to the outside of the imaging element without outputting image data without the flag to the outside. Furthermore, only the information regarding the flag may be output to the outside as the signal data.

Furthermore, as a seventh example B, the information processing unit 101 may detect a feature region in an image of microscopic observation, and add the flag to image data of only the feature region or image data including only the feature region and peripheral pixels. The flag may include data such as coordinates, operation time, and operation region. As an example of the detection of the feature region, for example, the visual field repeatedly observed by the user may be detected from the image analysis.

Similarly to the seventh example B described above, the image data or signal data with the flag may be output to the outside of the imaging element. Furthermore, the information processing unit 101 may generate image data of only a feature region detected as the feature region or image data including only this feature region and peripheral pixels, and output the image data of only the feature region or the image data including only this feature region and peripheral pixels and signal data to the outside of the imaging element.

Furthermore, as a seventh example C, in a case of observing a plurality of image frames, the information processing unit 101 images a moving image at a constant frame rate, and calculates a moving speed from an image change between visual fields (imaging frames). In a case where the moving speed is converted, the information processing unit 101 adds a flag to a frame with a change. The information processing unit 101 can reduce output image data by compressing image data other than image data with the flag.

Note that the seventh example according to the present technology can be applied by appropriately employing methods as necessary from the data acquisition methods described in the first example to the sixth example, and appropriately combining the methods as necessary.

### 2. Second Embodiment (Application Device)

The data acquisition device according to the present technology can be applied as various devices, and may be provided in various devices. Examples of the device include, but are not limited to, a cell culture apparatus, a microscopic observation device, a nucleic acid sequence analysis device, a biological tissue observation device, a biological sample observation device, and the like. The nucleic acid sequence analysis device may be, for example, a next generation sequencer (NGS, Next Generation Sequencer). This is as described in 1. above, and the description is also applicable to the present embodiment.

### 3. Third Embodiment (Data Acquisition Method)

The present technology also provides a data acquisition method including:
a signal acquisition step of acquiring image signals of a biological sample at two or more different time points; a feature amount extraction step of extracting a feature amount from the image signals;
a data generation step of generating data related to the biological sample on the basis of the feature amount; and
an output step of causing the data related to the biological sample to be output to an outside of the imaging element.

The method of the present technology may include an irradiation step of irradiating the biological sample with light before the signal acquisition step.

The present technology also provides a data acquisition method including:
a feature amount extraction step of extracting a feature amount from image signals acquired by imaging a biological sample by an imaging element at two or more different time points;
a data generation step of generating data related to the biological sample on the basis of the feature amount; and
an output step of causing the data related to the biological sample to be output to an outside of the imaging element.

The data acquisition method according to the present technology may include a determining step of determining the state of the biological sample on the basis of the feature amount.

The data acquisition method according to the present technology can also generate the data related to the biological sample using, for example, a learned model.

Furthermore, the data acquisition method according to the present technology can be executed by the above-described device (for example, the data acquisition device described in the above 1., and the like).

The biological sample observation method of the present technology can include the above-described data acquisition method. The biological sample observation method may be a microscopic observation method or a nucleic acid sequence analysis method.

### 4. Fourth Embodiment (Program)

The present technology also provides a program to be executed by a data acquisition device including an imaging element including:
a signal acquisition unit that acquires image signals of a biological sample at two or more different time points;
an information processing unit that extracts a feature amount from the image signals and generates data related to the biological sample on the basis of the feature amount; and
an output control unit that causes the data related to the biological sample to be output to an outside of the imaging element. The program is as described in the above 1. to 3., and the description also applies to the present embodiment.

In the feature amount extraction step, the feature amount is extracted from image signals of the biological sample at two or more different time points. The data generation step generates the data related to the biological sample on the basis of the feature amount. In the output step, the data related to the biological sample is output to the outside of the imaging element. In order to generate the data related to the biological sample, a learned model may be included and may be stored in a storage unit or the like outside the data acquisition device.

### 5. Fifth Embodiment (Biological Sample Observation System)

The present technology provides a biological sample observation system including:
a holding unit capable of holding a biological sample;
an irradiation unit that irradiates the biological sample with light; and
an imaging element including
a signal acquisition unit that acquires image signals of the biological sample at two or more different time points,
an information processing unit that extracts a feature amount from the image signals and generates data related to the biological sample on the basis of the feature amount, and
an output control unit that causes the data related to the biological sample to be output to an outside of the imaging element, in which
the signal acquisition unit, the information processing unit, and the output unit are arranged in a single chip.

The biological sample observation system may further include an incubator that stores the holding unit.

The biological sample observation system may be a microscopic observation system or a nucleic acid sequence analysis system.

The system is as described in the above 1. to 4., and the description also applies to the present embodiment.

Note that the present technology can also have the following configurations.

[1]

A data acquisition device including
an imaging element including
an acquisition unit that acquires image signals of a biological sample at two or more different time points,
an information processing unit that extracts a feature amount from the image signals and generates data related to the biological sample on the basis of the feature amount, and
an output control unit that causes the data related to the biological sample to be output to an outside of the imaging element, in which
the signal acquisition unit, the information processing unit, and the output unit are arranged in a single chip.
   [2]

The data acquisition device according to [1] above, in which
the signal acquisition unit has a configuration in which a plurality of pixels is arranged two-dimensionally, and
the imaging element is configured to image the biological sample through an objective lens.
   [3]

The data acquisition device according to [1] or [2], in which the information processing unit generates the data related to the biological sample using a learned model.

[4]

The data acquisition device according to any one of [1] to [3], in which
the information processing unit includes a feature amount extraction unit that acquires the feature amount and a state determination unit that determines a state of the biological sample on the basis of the feature amount, and
the information processing unit generates data related to a biological sample to be output on the basis of a determination result by the state determination unit.
   [5]

The data acquisition device according to any one of [1] to [4], in which the feature amount is any of a feature amount related to a cell culture, a feature amount related to a fertilized egg, a feature amount related to a sperm, a feature amount related to a nucleic acid, or a feature amount related to a biological tissue piece.

[6]

The data acquisition device according to any one of [1] to [5], in which the biological sample is one kind or two or more kinds selected from a cell culture, a fertilized egg, a sperm, a nucleic acid, and a biological tissue piece.

[7]

The data acquisition device according to any one of [1] to [6], in which the data related to the biological sample includes image data, alert data, flag data, or nucleic acid sequence data.

[8]

The data acquisition device according to any one of [1] to [7], in which
the biological sample includes a cell culture, and
the information processing unit determines whether a predetermined cell density is reached or a foreign substance is generated in the cell culture on the basis of the feature amount related to the cell culture.
   [9]

The data acquisition device according to any one of [1] to [7] and [8], in which
the biological sample includes a cell culture, and
the information processing unit generates image data of the cultured cell on the basis of the feature amount related to the cell culture.
   [10]

The data acquisition device according to any one of [1] to [7], in which
the biological sample includes a fertilized egg, and
the information processing unit determines whether a predetermined division process has been reached on the basis of the feature amount related to the fertilized egg.
   [11]

The data acquisition device according to any one of [1] to [7] and [10], in which
the biological sample includes a fertilized egg, and
the information processing unit generates image data of the fertilized egg on the basis of a feature amount related to the fertilized egg.
   [12]

The data acquisition device according to any one of [1] to [7], in which
the biological sample includes sperm, and
the information processing unit determines a state of the sperm on the basis of the feature amount related to the sperm.
   [13]

The data acquisition device according to any one of [1] to [7] and [12], in which
the biological sample includes sperm, and
the information processing unit generates image data of the sperm on the basis of the feature amount related to the sperm.
   [14]

The data acquisition device according to any one of [1] to [7], in which
the biological sample includes a nucleic acid, and
the information processing unit generates sequence data of the nucleic acid on the basis of the feature amount related to the nucleic acid.
   [15]

A data acquisition method, including:
a feature amount extraction step of extracting a feature amount from image signals acquired by imaging a biological sample by an imaging element at two or more different time points;
a data generation step of generating data related to the biological sample on the basis of the feature amount; and
an output step of causing the data related to the biological sample to be output to an outside of the imaging element.
   [16]

A biological sample observation system including:
a holding unit capable of holding a biological sample;
an irradiation unit that irradiates the biological sample with light; and
an imaging element including a signal acquisition unit that acquires image signals of the biological sample at two or more different time points, an information processing unit that extracts a feature amount from the image signals and generates data related to the biological sample on the basis of the feature amount, and an output control unit that causes the data related to the biological sample to be output to an outside of the imaging element, in which
the signal acquisition unit, the information processing unit, and the output unit are arranged in a single chip.
   [17]

The biological sample observation system according to above, further including an incubator that stores the holding unit.

[18]

The biological sample observation system according to [16] or [17] above, in which the biological sample observation system is a microscopic observation system.

The biological sample observation system according to [16] above, in which the biological sample observation system is a nucleic acid sequence analysis system.

### REFERENCE SIGNS LIST

1 Data processing device
100 Imaging element
101 Information processing unit
102 Feature amount extraction unit
103 State determination unit
104 Recognition processing unit
105 Image generation unit
110 Signal acquisition unit (imaging unit)
120 Imaging processing unit
150 Output control unit

1. A data acquisition device comprising
an imaging element including
a signal acquisition unit that acquires image signals of a biological sample at two or more different time points,
an information processing unit that extracts a feature amount from the image signals and generates data related to the biological sample on a basis of the feature amount, and
an output control unit that causes the data related to the biological sample to be output to an outside of the imaging element, wherein
the signal acquisition unit, the information processing unit, and the output unit are arranged in a single chip.

2. The data acquisition device according to claim 1, wherein
the signal acquisition unit has a configuration in which a plurality of pixels is arranged two-dimensionally, and
the imaging element is configured to image the biological sample through an objective lens.

3. The data acquisition device according to claim 1, wherein the information processing unit generates the data related to the biological sample using a learned model.

4. The data acquisition device according to claim 1, wherein
the information processing unit includes a feature amount extraction unit that acquires the feature amount and a state determination unit that determines a state of the biological sample on a basis of the feature amount, and
the information processing unit generates data related to a biological sample to be output on a basis of a determination result by the state determination unit.

5. The data acquisition device according to claim 1, wherein the feature amount is any of a feature amount related to a cell culture, a feature amount related to a fertilized egg, a feature amount related to a sperm, a feature amount related to a nucleic acid, or a feature amount related to a biological tissue piece.

6. The data acquisition device according to claim 1, wherein the biological sample is one kind or two or more kinds selected from a cell culture, a fertilized egg, a sperm, a nucleic acid, and a biological tissue piece.

7. The data acquisition device according to claim 1, wherein the data related to the biological sample includes image data, alert data, flag data, or nucleic acid sequence data.

8. The data acquisition device according to claim 1, wherein
the biological sample includes a cell culture, and
the information processing unit determines whether a predetermined cell density is reached or a foreign substance is generated in the cell culture on a basis of the feature amount related to the cell culture.

9. The data acquisition device according to claim 1, wherein
the biological sample includes a cell culture, and
the information processing unit generates image data of the cultured cell on a basis of the feature amount related to the cell culture.

10. The data acquisition device according to claim 1, wherein
the biological sample includes a fertilized egg, and
the information processing unit determines whether a predetermined division process has been reached on a basis of the feature amount related to the fertilized egg.

11. The data acquisition device according to claim 1, wherein
the biological sample includes a fertilized egg, and
the information processing unit generates image data of the fertilized egg on a basis of a feature amount related to the fertilized egg.

12. The data acquisition device according to claim 1, wherein
the biological sample includes sperm, and
the information processing unit determines a state of the sperm on a basis of the feature amount related to the sperm.

13. The data acquisition device according to claim 1, wherein
the biological sample includes sperm, and
the information processing unit generates image data of the sperm on a basis of the feature amount related to the sperm.

14. The data acquisition device according to claim 1, wherein
the biological sample includes a nucleic acid, and
the information processing unit generates sequence data of the nucleic acid on a basis of the feature amount related to the nucleic acid.

15. A data acquisition method, comprising:
a feature amount extraction step of extracting a feature amount from image signals acquired by imaging a biological sample by an imaging element at two or more different time points;
a data generation step of generating data related to the biological sample on a basis of the feature amount; and
an output step of causing the data related to the biological sample to be output to an outside of the imaging element.

16. A biological sample observation system comprising:
a holding unit capable of holding a biological sample;
an irradiation unit that irradiates the biological sample with light; and
an imaging element including a signal acquisition unit that acquires image signals of the biological sample at two or more different time points, an information processing unit that extracts a feature amount from the image signals and generates data related to the biological sample on a basis of the feature amount, and an output control unit that causes the data related to the biological sample to be output to an outside of the imaging element, wherein
the signal acquisition unit, the information processing unit, and the output unit are arranged in a single chip.

17. The biological sample observation system according to claim 16, further comprising
an incubator that stores the holding unit.

18. The biological sample observation system according to claim 16, wherein
the biological sample observation system is a microscopic observation system.

19. The biological sample observation system according to claim 16, wherein
the biological sample observation system is a nucleic acid sequence analysis system.
